(19) United States
(12) Patent Application Publication
(10) Pub. No.: US 2010/0113619 A1
(43) Pub. Date: May 6, 2010

(54) HIGH-AMYLOSE SODIUM CARBOXYMETHYL STARCH SUSTAINED RELEASE EXCIPIENT AND PROCESS FOR PREPARING THE SAME

(75) Inventors: Fabien Brouillet, Bessan (FR); Bernard Bataille, Saint-Gely-du-Fesc (FR); Gilles Baylac, Jacou (FR); Louis Cartilier, Beaconsfield (CA)

Correspondence Address: THE NATH LAW GROUP, 112 South West Street, Alexandria, VA, 22314, US

(73) Assignee: UNIVERSITÉ DE MONTRÉAL, Montreal, QC (CA)

(21) Appl. No.: 12/451,907

(22) PCT Filed: Jun. 5, 2008
(86) PCT No.: PCT/CA2008/001089

§ 371 Date: Dec. 4, 2009

(30) Foreign Application Priority Data
Jun. 7, 2007 (CA) 2,590,821

(51) Int. Cl. A61K 47/36; C08B 31/00; A61K 9/20

(52) U.S. Cl. 514/778; 536/102

(57) ABSTRACT

A process for obtaining a spray-dried high amylose sodium carboxymethyl starch comprising a major fraction of amorphous form and optionally a minor fraction of crystalline V form, is provided. The process comprises providing an amorphous pregelatinized high amylose sodium carboxymethyl starch (HASCA); dispersing the amorphous pregelatinized HASCA in a solution comprising water and at least one first pharmaceutically acceptable organic solvent miscible with water and suitable for spray-drying; and spray-drying the dispersion to obtain the spray-dried HASCA comprising a major fraction of amorphous form and optionally a minor fraction of crystalline V form, in the form of a powder. Also provided is a spray-dried HASCA sustained-release excipient. This excipient is useful for preparing a tablet for the sustained-release of at least one drug.

### FIELD OF THE INVENTION

The present invention relates to a sustained-release excipient for drug formulation. More specifically, the invention relates to a high-amylose sodium carboxymethyl starch as a pharmaceutical sustained drug-release tablet excipient. The invention also relates to a process for preparing such excipient.

### DESCRIPTION OF THE PRIOR ART

### Drug Controlled Release, Matrix Tablets and Polymers

For many years, increased attention has been given to drug administration characteristics, which has led to the development of new pharmaceutical dosage forms allowing control of drug release. Among the many oral dosage forms that can be used for sustained drug-release, tablets are of major interest in the pharmaceutical industry because of their highly efficient manufacturing technology.

Matrix tablets obtained by direct compression of a mixture of a drug with a polymer would be the simplest way to achieve orally a controlled release of the active ingredient. Of course, these tablets should also show good mechanical qualities (i.e. tablet hardness and resistance to friability) in order to meet the manufacturing process requirements and the subsequent handling and packaging requirements.

Furthermore, the matrix polymers should be easily obtained, biocompatible and non-toxic, with the proviso that biodegradable synthetic polymers have the disadvantage of a possible toxicity following absorption of the degraded products.

Starches and modified starches are examples of polymers currently used in the food and pharmaceutical industries. Various starch-modification methods, either chemical, physical, enzymatic or a combination thereof, are employed to create new starch products with specific or improved properties. Starch is considered a good candidate for chemical reaction/transformation because of its composition, i.e. mixture of amylose and amylopectin, two glucose polymers presenting three hydroxyl groups available as chemically-active, functional entities. Oxidation, ethoxylation and carboxymethylation are some of the modifications commonly deployed to prepare starch derivatives.

### Starches and Modified Starches

Unmodified, modified, derivatized and cross-linked starches have been proposed as binders, disintegrants or fillers in tablets [Short et al., U.S. Pat. No. 3,622,677 and No. 4,072,535; Trubiano, U.S. Pat. No. 4,369,308; McKee, U.S. Pat. No. 3,034,911], but no controlled release properties have been described. More particularly, carboxymethylstarch has been disclosed as a tablet disintegrant [McKee, U.S. Pat. No. 3,034,911]. Mehta, A. et al. [U.S. Pat. No. 4,904,476] disclosed the use of sodium starch glycolate as a disintegrant. These two patents relate to carboxymethylstarch having a low content in amylose but also disclose a disintegrant, which is the opposite of a sustained-release system. One knows today that high amylose content is an essential feature to obtain sustained drug-release properties [see Cartilier, L. et al., U.S. Pat. No. 5,879,707, Substituted amylose as a matrix for sustained drug release].

Some works have disclosed the use of physically- and/or chemically-modified starches for sustained drug-release. The authors of these papers have presented the usual types of starches, i.e. those containing low amounts of amylose, and have not even mentioned the role of amylose, nor amylose itself [Nakano, M. et al., Chem. Pharm. Bull., 35, 4346-4350 (1987); Van Aerde, P. et al., Int. J. Pharm., 45, 145-152 (1988)]. Some works have even attributed a negative role to amylose present in thermally-modified starches used in sustained drug-release tablets [Hermann, J. et al., Int. J. Pharm., 56, 51-63 & 65-70 (1989) and Int. J. Pharm., 63, 201-205 (1990)]. Staniforth, J. et al., [U.S. Pat. No. 5,004,614] disclose a controlled-release device with an impermeable coating that is substantially impermeable to the entrance of an environmental fluid and substantially impermeable to the exit of the active agent during a dispensing period and having an orifice for drug release. Cross-linked or uncross-linked sodium carboxymethylstarch is proposed among other materials for the coating. The coated controlled-release system described herein is totally different from a matrix tablet when considering the structural aspects and the release mechanisms involved. Also, the presence of an orifice through the coating is necessary. U.S. Pat. No. 5,004,614 also requires the coating to be impermeable to aqueous environment, such being contrary to a hydrophilic matrix system which implies necessarily that water penetrates the tablet. Finally, U.S. Pat. No. 5,004,614 does not mention the necessity of having a high content in amylose.

### Physically-Modified Amylose and “Short Chain Amylose”

Physical modifications of amylose for pharmaceutical formulations have also been disclosed: non-granular amylose as a binder-disintegrant [Nichols et al., U.S. Pat. No. 3,490,742], and glassy amylose as a coating for oral, delayed-release composition due to enzymatic degradation of the coating into the colon [Alwood et al., U.S. Pat. No. 5,108,758]. These patents are not related to high-amylose carboxymethylamylose as a matrix excipient for sustained drug-release.

Wai-Chiu C. et al. [Wai-Chiu et al., U.S. Pat. No. 5,468,286] disclosed a starch binder and/or filler useful in manufacturing tablets, pellets, capsules or granules. The tablet excipient is prepared by enzymatically debranching starch with alpha-1,6-D-glucanohydrolase to yield at least 20% by weight of “short chain amylose”, i.e. linear chains containing from about 5 to 65 glucose units. No controlled release properties are claimed for this excipient. Thus, starch with a high content of amylopectin is obviously preferred, and amylose is rejected as being unsuitable because debranching is impossible since it has no branching. The role of amylose is not only ignored but also considered negatively. In connection with this reference, it must also be emphasized that “short-chain amylose” does not exist.

### Cross-Linked Amylose

Several patents relate to the use of cross-linked amylose in tablets for drug controlled-release or as a binder-disintegrant in certain cases [Mateescu, M. et al., U.S. Pat. No. 5,456,921; Mateescu, M. et al., U.S. Pat. No. 5,603,956; Cartilier, L. et al., U.S. Pat. No. 5,616,343; Dumoulin, Y. et al., U.S. Pat. No. 5,807,575; Chouinard, F. et al., U.S. Pat. No. 5,885,615; Cremer, K. et al., U.S. Pat. No. 6,238,698].

Lenaerts, V. et al. [U.S. Pat. No. 6,284,273] disclose cross-linked high amylose starch rendered resistant to amylase. Such amylase resistant starches are obtained by co-cross-linking high amylose starch with polyols. Suitable agents that could be used as additives to high amylose starch for controlled release prior to cross-linking of the high amylose starch include, but are not limited to, polyvinyl alcohol, beta-(1-3) xylan, xanthan gum, locust bean gum and guar gum.

Lenaerts, V. et al. [U.S. Pat. No. 6,419,957] disclose cross-linked high amylose starch having functional groups as a matrix for the slow release of pharmaceutical agents. This matrix tablet excipient is prepared by a process comprising the steps of: (a) reacting high amylose starch with a cross-linking agent cross-linked at a concentration of about 0.1 g to about 40 g of cross-linking agent per 100 g of high amylose starch to afford cross-linked amylose; and (b) reacting the cross-linked high amylose starch with a functional group-attaching reagent at a concentration of about 75 g to about 250 g of functional group-attaching reagent per 100 g of cross-linked amylose to afford the cross-linked amylose having functional group.

Lenaerts, V. et al. [U.S. Pat. No. 6,607,748] disclose cross-linked high amylose starch for use in controlled-release pharmaceutical formulations and processes for its manufacture. Such cross-linked high amylose starch is prepared by (a) cross-linking and chemical modification of high amylose starch, (b) gelatinization, and (c) drying to obtain a powder of said controlled-release excipient.

Lenaerts, V. et al. [WO 2004/038428 A2] disclose cross-linked high amylose starch for use in solid dosage formulations having a core with tramadol.HCl dispersed in a first controlled-release matrix from which release of the agent is relatively slow and a coat formed over the core and having the agent dispersed in a second controlled-release matrix from which release of the drug is relatively fast. The first matrix is a cross-linked high amylose starch and the second matrix can be a mixture of polyacetate and polyvinylpyrrolidone. Cross-linked high amylose starch is prepared according to the process disclosed in U.S. Pat. No. 6,607,748.

According to their authors, all these patents disclose only cross-linked amylose and some of its variants, which are to be distinguished from linearly substituted amylose, which does not show any chemical cross-linking.

### Substituted Amylose

Substituted amylose (SA) has been introduced as a promising pharmaceutical excipient for sustained drug-release. U.S. Pat. No. 5,879,707 describes SA matrix tablets which have been prepared by direct compression, i.e. dry mixing of drug and SA polymers, followed by compression, which is the easiest way to manufacture an oral dosage form [see also Chebli, C. et al. in “Substituted amylose as a matrix for sustained drug release”, Pharm. Res. 1999, 16 (9), 1436-1440].

High-amylose corn starch, containing 70% of amylose chains and 30% of amylopectin, has been tested for the production of SA polymers by an etherification process. These polymers are referred to as SA,R-n, where R defines the substituent and n represents the degree of substitution (DS) expressed as the ratio of mole of substituent/kg of amylose [see U.S. Pat. No. 5,879,707 and Chebli, C. et al., Pharm. Res. 1999, 16 (9), 1436-1440]. First, a range of substituents such as 1,2-epoxypropanol (or glycidol=G), 1,2-epoxybutane, 1,2-epoxydecane and 1-chlorobutane, were investigated. SA,G polymers and especially SA,G-2.7 demonstrated interesting properties as excipients for controlled drug-release systems. SA,G-2.7 matrices allowed nearly constant drug-release. Moreover, sustained drug-release matrix systems based on SA,G technology presented large ranges for drug-loading, drug solubility and tablet weight [see U.S. Pat. No. 5,879,707 and Chebli, C. et al. in “Effect of some physical parameters on the sustained drug-release properties of substituted amylose matrices. Int. J. Pharm. 2000, 193 (2), 167-173]. Release time is directly proportional to tablet weight (TW) for tablets containing 10% of acetaminophen. Another advantage of this excipient is that there is no significant influence of compression forces, ranging from 0.5 to 5.0 tons/cm², on the release properties of SA,G-n polymers with a DS greater than 1.5.

In contrast to pre-gelatinized starches known for their poor binding properties, as described by Rahmouni, M. et al. in “Influence of physical parameters and lubricants on compaction properties of granulated and non-granulated cross-linked high amylose starch”, Chem. Pharm. Bull. 2002, 50 (9), 1155-1162 or by Hancock, B. et al. in “The powder flow and compact mechanical properties of two recently developed matrix-forming polymers”, J. Pharm. Pharmacol. 2001, 53 (9), 1193-1199, SA,G polymers have shown good compression behaviour, which results in unusually high crushing strength values comparable to those of microcrystalline cellulose tablets, a reference among binders/fillers [see U.S. Pat. No. 5,879,707]. The high crushing strength values obtained for these tablets are due to an unusual sintering process occurring during tableting, although the tablet's external layer goes only through densification, deformation and partial melting [see Moghadam, S. H. et al. in “Substituted amylose matrices for oral drug delivery”, Biomed. Mater 2007, 2, S71 -S77].

Reacting high amylose starch with sodium chloroacetate/chloroacetic acid in place of non-ionic substituents has been proposed for excipients more readily acceptable by regulatory agencies [see Canadian Patent Application No. 2,591,806 and Ungur, M. et al., “The evaluation of carboxymethylamylose for oral drug delivery systems: from laboratory to pilot scale”, 3^{rd} International Symposium on Advanced Biomaterials/Biomechanics, Montreal, Canada, 2005; Book of Abstracts, p. 271]. Indeed, carboxymethyl starch containing low amounts of amylose already serves as a disintegrating agent in immediate-release tablets [Bolhuis, G. K. et al., “On the similarity of sodium starch glycolate from different sources”, Drug. Dev. Ind. Pharm. 1986, 12 (4), 621-630; and Edge, S. et al., “Sodium starch glycolate”, in Handbook of Pharmaceutical Excipients, 5th ed.; Rowe, R. C.; Sheskey, P. J.; Owen, S. C., Eds. Pharmaceutical Press/American Pharmacists Association: London-Chicago, 2005; pp 701-704].

In contrast, high-amylose sodium carboxymethyl starch (HASCA) has been recently suggested as a suitable material for oral matrix tablets [see Cartilier, L., Canadian Patent Application No. 2,591,806; and Ungur, M. et al., “The evaluation of carboxymethylamylose for oral drug delivery systems: from laboratory to pilot scale”, 3^{rd} International Symposium on Advanced Biomaterials/Biomechanics, Montreal, Canada, 2005; Book of Abstracts, p. 271]. These tablets can be advantageously improved by the addition of electrolytes as the polymer is ionic. Such addition permits the integrity of the swollen matrix tablets to be maintained when they are immersed in a medium undergoing pH changes simulating the pH evolution of the environment surrounding an oral pharmaceutical dosage form transiting along the gastrointestinal tract while allowing controlled and sustained drug-release with a remarkably close-to-linear release profile.

There is thus a need for an industrial-scale process for preparing high-amylose sodium carboxymethyl starch (HASCA) as a pharmaceutical sustained drug-release tablet excipient.

There is a need for an economical industrial and environmentally safe process for producing a sustained-drug release HASCA excipient for matrix tablets.

### SUMMARY OF THE INVENTION

The present invention provides an original process for transforming amorphous pregelatinized HASCA into a suitable sustained drug-release excipient for matrix tablets. The process of the present invention has the advantage to be economical industrially and environmentally safe.

The present invention also provides a pharmaceutical excipient having sustained-release properties obtained by the process of the invention. Such excipient is useful as a matrix for tablets for oral administration.

In one aspect, the present invention relates to a process for obtaining a spray-dried high amylose sodium carboxymethyl starch comprising a major fraction of amorphous form and optionally a minor fraction of crystalline V form. The process comprises the following steps:
a) providing an uncross-linked amorphous pregelatinized high amylose sodium carboxymethyl starch (HASCA);
b) dispersing the uncross-linked amorphous pregelatinized high amylose sodium carboxymethyl starch in a solution comprising water and at least one first pharmaceutically acceptable organic solvent miscible with water and suitable for spray-drying; and
c) spray-drying the dispersion to obtain the spray-dried high amylose sodium carboxymethyl starch comprising a major fraction of amorphous form and optionally a minor fraction of crystalline V form, in the form of a powder.

In an embodiment, the uncross-linked amorphous pregelatinized high amylose sodium carboxymethyl starch provided in step a) of the process of the invention is beforehand dried by a roller-dryer.

In another embodiment, an amount of second pharmaceutically acceptable organic solvent(s) miscible with water and suitable for spray-drying is added to the heated dispersion before the spray-drying step. For instance, the addition of a second solvent may be useful to reduce the viscosity of the dispersion. The second solvent(s) added at this optional step may be different or identical to the first solvent(s) used to form the dispersion of the HASCA.

The organic solvents used in the process according to the invention should be pharmaceutically acceptable and miscible with water. These solvents should also be suitable for spray-drying methods. The expression “pharmaceutically acceptable solvent” means that the solvent is useful in preparing a pharmaceutical composition that is generally non-toxic and is not biologically undesirable. Thus, pharmaceutically acceptable solvents include solvents which are acceptable for veterinary use and/or human pharmaceutical use. A “water-miscible solvent” refers to a solvent for which the volume of the aqueous phase used in the process is sufficient to dissolve the total amount of organic solvent used. Accordingly, the organic solvent must be at least partially water-miscible.

A combination of organic solvents could also be used in the process according to the invention. Examples of solvents, to be used in the process of the present invention are ethanol, n-propanol, isopropanol, ter-butanol or acetone. In an embodiment, the solvent is ethanol or isopropanol, or a mixture thereof.

The relative quantities of water and organic solvent(s) in the initial solution (step a)) may vary but keeping in mind that the process is intended to be environmentally safe, thus using the less organic solvent as possible. Thus, the water to organic solvent(s) weight ratio in the initial solution is generally above 1.

The HASCA used according to the invention includes a high concentration of amylose compared to traditional starch. The amylose is an amylose having a long chain consisting of more than 250 glucose units (between about 1,000 and about 5,000 units), joined by alpha-1,4-D glucose links, in a linear sequence. In an embodiment, the HASCA includes at least about 50 weight % amylose. For instance, it includes at least about 60 weight % amylose. In another embodiment the HASCA includes at least about 70 weight % amylose. Moreover, the substitution degree (DS) (number of moles of substituent/number of moles of anhydroglucose) of the HASCA is for instance comprised between about 0.005 and about 0.070. In an embodiment, the DS is about 0.045.

The term “about” used in the context of the present invention is intended to represent a variation of ±10% of the values provided herein.

In another aspect, the present invention relates to a spray-dried high amylose sodium carboxymethyl starch (spray-dried HASCA) sustained-release excipient comprising a major fraction of amorphous form and optionally a minor fraction of crystalline V form obtained by the process of the invention as described above.

The invention further relates to a spray-dried high amylose sodium carboxymethyl starch sustained-release excipient comprising a major fraction of amorphous form and optionally a minor fraction of crystalline V form, wherein the excipient is obtained by spray-drying a dispersion of an uncross-linked amorphous pregelatinized high amylose sodium carboxymethyl starch in a solution comprising water and ethanol, or isopropanol or a mixture thereof, the amorphous pregelatinized high amylose sodium carboxymethyl starch comprising at least about 60 weight % of amylose and having a substitution degree of about 0.045.

In a further aspect, the invention relates to the use of the spray-dried high amylose sodium carboxymethyl starch sustained-release excipient as defined hereinabove in the preparation of a tablet for sustained-release of at least one drug.

In another aspect the invention provides a tablet for sustained-release of at least one drug comprising the spray-dried high amylose sodium carboxymethyl starch sustained-release excipient as defined hereinabove and at least one drug.

The spray-dried HASCA sustained-release excipient may be used alone in the tablet or in combination with at least one electrolyte. For instance, the electrolytes useful in the present invention may be calcium chloride, potassium chloride, sodium chloride, magnesium chloride, sodium sulfate, zinc sulphate or aluminium sulphate. Other possible electrolytes may be citrate, tartrate, maleate, acetate, phosphate (dibasic and monobasic), glutamate, carbonate salts, which are soluble or partially soluble in aqueous media having a pH similar to the ones of the GI tract. Alternatively, the electrolytes may be calcium or ferrous gluconate, calcium lactate, aminoacids derivates such as arginine hydrochloride, citric acid, tartaric acid, maleic acid, or glutamic acid. The electrolyte may also be another excipient, a drug or mixture thereof. In an embodiment, the electrolyte is sodium chloride or potassium chloride.

The drugs which may be used in the tablet of the invention include drugs qualified as very soluble, freely soluble, soluble, sparingly soluble, slightly soluble and very slightly soluble in conformity with the nomenclature of the United States Pharmacopeia [“The United States Pharmacopeia XXIII-The National Formulary XVIII”, 1995. See Table page 2071 entitled “Description and Solubility”].

The invention and its advantages will be better understood upon reading the following non-restrictive detailed description and examples, with reference being made to the accompanying drawings.

### BRIEF DESCRIPTION OF THE FIGURES

FIG. 1 shows powder X-ray diffraction patterns of different HASCA samples produced by spray-drying. The spectra have been staggered for clarity purpose.

FIG. 2 shows a scanning electron microscope picture of amorphous pregelatinized HASCA particles obtained by roller-dryer.

FIG. 3 shows a scanning electron microscope picture of SD-A HASCA particles.

FIG. 4 shows a scanning electron microscope picture of SD-D HASCA particles.

FIG. 5 is a diagram showing the influence of % w/w HASCA-I of initial hydro-alcoholic HASCA suspensions on SD HASCA tablet hardness for different water concentrations of the starting hydro-alcoholic solution (: 65.22% w/w WATER; ▪: 74.47% w/w WATER).

FIG. 6 is a diagram showing the influence of HASCA concentration in spray-drying solution (% w/w HASCA-II) on SD HASCA tablet hardness.

FIG. 7 is a diagram showing the influence of % w/w WATER of the starting hydro-alcoholic solution on SD HASCA tablet hardness for different weights of HASCA powder dispersed in 80 g of hydro-alcoholic solution (▪: 12 g HASCA; ♦: 10 g HASCA).

FIG. 8 is a diagram presenting the cumulative percentage of acetaminophen released in vitro from optimized SD HASCA matrices (32.5% of SD HASCA, 40% of acetaminophen, and 27.5% of NaCl) in standard pH gradient conditions (▴: SD-A, ◯: SD-D).

FIG. 9 is a diagram showing the influence of tablet weight (TW) on tablet thickness (TT) of SD HASCA matrix tablets containing 40% acetaminophen and 27.5% NaCl under different CFs (▴: 1 ton/cm², ▪: 1.5 ton/cm², ♦: 2.5 tons/cm²).

FIG. 10 is a diagram showing the effect of compression force (CF) on acetaminophen release from SD HASCA tablets containing 40% acetaminophen and 27.5% NaCl (600-mg tablets, CF 1 ton/cm²: ◯; 600-mg tablets, CF 1.5 tons/cm²: □; 600-mg tablets, CF 2.5 tons/cm²: Δ; 400-mg tablets, CF 1 ton/cm²: ; 400-mg tablets, CF 1.5 tons/cm²: ▪; 400-mg tablets, CF 2.5 tons/cm²: ▴).

FIG. 11 is a diagram showing the effect of TW on % acetaminophen release from 300-mg (dotted line), 400-mg (dashed line) and 600-mg (continuous line) SD HASCA matrix tablets containing 40% acetaminophen and 27.5% NaCl.

FIG. 12 is a diagram showing the effect of TW on acetaminophen T25% (▴), T50% () and T95% (♦) release from SD HASCA tablets containing 40% acetaminophen and 27.5% NaCl.

FIG. 13 is a diagram showing the influence of drug-loading on acetaminophen release from 600-mg SD HASCA tablets compressed at 2.5 tons/cm² containing 10% acetaminophen (dashed line) or 40% acetaminophen (continuous line).

FIG. 14 is a diagram showing the effect of NaCl particle size distribution on acetaminophen release from 600-mg SD HASCA tablets compressed at 2.5 tons/cm² containing 40% acetaminophen and 27.5% NaCl (300-250-μm fraction: dotted line, 600-425-μm fraction: dashed line and 600-125-μm fraction: continuous line).

FIG. 15 presents pictures of typical 600-mg SD HASCA tablet matrices (40% acetaminophen, 27.5% NaCl, 32.5% HASCA), compressed at 2.5 tons/cm², after immersion in a pH gradient simulating the pH evolution of the gastrointestinal tract (pH 1.2 for 1 hour, pH 6.8 for 3 hours, and pH 7.4 until the end of the dissolution test): a) 2 hours of immersion b) 4 hours of immersion c) 8 hours of immersion d) 13 hours of immersion e) 16 hours of immersion and f) 22 hours of immersion.

FIG. 16 is a diagram showing the cumulative percentage of acetaminophen released in vitro in a pH gradient medium from SD HASCA tablet matrices weighing 500 mg and compressed at 2.5 tons (A: acetaminophen 30%, SD HASCA 70%; B: acetaminophen 30%, SD HASCA 55%, NaCl 15%; C: acetaminophen 30%, SD HASCA 55%, KCl 15%).

FIG. 17 is a diagram showing the effect of the solvent used in the spray-drying process on % acetaminophen release from 600-mg P7 SD HASCA matrix tablets containing 40% acetaminophen and 27.5% NaCl (dotted line=ethanol; continuous line=isopropanol).

FIG. 18 is a diagram showing the effect of NaCl content on % acetaminophen release from 600-mg P6 SD HASCA matrix tablets containing 40% acetaminophen (dotted line=27.5% NaCl; continuous line=22.5% NaCl).

FIG. 19 is a diagram showing the % acetaminophen release from 500-mg P6 SD HASCA matrix tablets containing 40% acetaminophen and 17.5% NaCl.

### DETAILED DESCRIPTION OF THE INVENTION

### Preliminary Considerations

The first laboratory scale batches of non-ionic SA polymers were prepared by reacting the substituent and high amylose starch in a heated alkaline medium. After neutralization of the suspension, the resultant gel was filtered and washed with water and acetone. The powder product was exposed overnight to air, allowing to collect the excipient in a readily-compressible powder form [U.S. Pat. No. 5,879,707]. HASCA was then produced according to a similar lab-scale process [Canadian Patent Application No. 2,591,806 and Ungur, M. et al., “The evaluation of carboxymethylamylose for oral drug delivery systems: from laboratory to pilot scale”, 3^{rd} International Symposium on Advanced Biomaterials/Biomechanics, Montreal, Canada, 2005; Book of Abstracts, p. 271]. SA,G-2.7 and HASCA produced at the lab scale both demonstrated excellent binding and sustained drug-release properties.

### The Problem

HASCA was obtained on a pilot scale using a drying method without organic solvents. However, the HASCA appeared to be unsuitable for tableting and sustained drug-release. In order to obtain a dry powder presenting the required binding and sustained drug-release properties, the dry powder of pilot-scale HASCA was thus dispersed in hot water, then precipitated with ethanol using the laboratory process, as described in U.S. Pat. No. 5,879,707 or Chebli, C. et al., Pharm. Res. 1999, 16 (9), 1436-1440, though the original process used acetone to precipitate SA polymers. The results are presented in Canadian Patent Application No. 2,591,806 and Ungur, M. et al., “The evaluation of carboxymethylamylose for oral drug delivery systems: from laboratory to pilot scale”, 3^{rd} International Symposium on Advanced Biomaterials/Biomechanics, Montreal, Canada, 2005; Book of Abstracts, p. 271.

However, the main drawback of the above method, i.e. precipitation by a non-solvent, is that very high volumes of organic solvent are needed to recover the product, yielding 1 part of solid recovered for up to 30 parts or more of ethanol. Such may be considered redhibitory in an environmental and industrial point of view.

### The Solution

Two main functions of the non-solvent may be distinguished: first, precipitation and crystallization, if any of HASCA, and, secondly, the removal of residual water to give a suitable dry powder. The first step is to dissolve the macromolecules. In the case of amylose, the macromolecules can be dispersed at a very low concentration in hot water [Whittam, M. A. et al., Aqueous dissolution of crystalline and amorphous amylose-alcohol complexes, Int. J. Biol. Macromol. 1989, 11 (6), 339-344; Yamashita, Y. et al., Single crystals of amylose V complexes. II. Crystals with 7₁ helical configuration, J. Polym. Sci.: Part A-2: Polym. Phys. 1966, 4 (2), 161-171; Booy, F. P. et al., Electron diffraction study of single crystals of amylose complexed with n-butanol, Biopolymers 1979, 18 (9), 2261-2266]. Then, the polymer is precipitated by a non-solvent addition. The problem with highly diluted solutions is that they require very high quantities of non-solvent to precipitate and collect a dry powder. Increasing the starch concentration in the solution may solve the problem. However, due to the presence of its hydroxyl groups, amylose in aqueous solution forms a gel through hydrogen-bonding. Thus, raising the starch concentration in water heightens the apparent viscosity of the solution and the gel formation of starch [McGrane, S. J. et al., The role of hydrogen bonding in amylose gelation, Starch/Stärke 2004, 56 (3-4), 122-131]. A way to overcome this problem is to employ an organic solvent or water/organic solution as medium to limit the formation of a viscous starch paste [Tijsen, C. J. et al, Optimisation of the process conditions for the modification of starch, Chem. Eng. Sci. 1999, 54 (13-14), 2765-2772; Tijsen, C. J. et al., An experimental study on the carboxymethylation of granular potato starch in non-aqueous media, Carbohyd. Polym. 2001, 45 (3), 219-226; Tijsen, C. J. et al., Design of a continuous process for the production of highly substituted granular carboxymethyl starch, Chem. Eng. Sci. 2001, 56 (2), 411-418]. Various organic liquids such as ethanol and isopropanol have been tested. It has been proposed that alcohol disrupts the amylose gel structure by bonding to hydroxyl groups on starch molecules. Unlike water-bonding, this binding is terminal and produces no connectivity between amylose molecules, reducing the apparent viscosity of the solution and resulting in amylose precipitation at high-alcohol concentrations [McGrane, S. J. et al., The role of hydrogen bonding in amylose gelation, Starch/Stärke 2004, 56 (3-4), 122-131].

The present invention proposes a more economical industrial production of HASCA. The process of the present invention is designed to transform, by spray-drying (SD), amorphous pregelatinized HASCA into a suitable sustained drug-release excipient for matrix tablets, while drastically decreasing ethanol quantities.

The present inventors had previously observed that 1) X-ray diffraction results of lab-scale batches, which were used as sustained-release matrices, showed the presence of a minor fraction of a substituted amylose V-form dispersed in a continuous amorphous phase and 2) pilot-scale HASCA obtained as a pregelatinized amorphous powder did not show any binding or sustained-release properties. In view of these observations, it was first thought that the V-form was necessary to obtain a suitable, sustained drug-release excipient (see Example 3b). However, further results surprisingly showed that in fact this V-form was not necessary to obtain sustained drug-release properties, but even decreased the binding properties of SD HASCA. Anyway, a delicate equilibrium had to be maintained between: a) adequately dispersing and/or dissolving HASCA to not allow crystalline re-arrangement of a fraction of HASCA shifting from the amorphous state to a V form, b) avoiding a too-high increase in viscosity to maintain acceptable SD conditions, and c) avoiding unfavourable HASCA gel formation and/or crystallization occurring before the SD process as the presence of a carboxylic function on glucosidic units of HASCA dramatically influences the gel-forming process through strong hydrogen-bonding. Furthermore, even if SD appears, at first glance, to be a practical method to easily remove large quantities of water from a pharmaceutical product, it is not evident that methods and results, if any, obtained for native starches and starch derivatives differing in the nature of their substituents and/or amylose concentration could be directly applied to the SD of HASCA. Experiments are thus necessary in case of processes implying a peculiar thermal treatment and fast rates of drying, particularly when the amorphous/crystalline state is of essence in achieving good tableting and sustained drug-release properties.

Thus, in a first step, hydro-alcoholic solutions with different water/ethanol ratios and HASCA powder concentrations were prepared. Water concentration had to remain as low as possible to limit dissolution of the starch, thus avoiding a too-high viscosity hindering agitation and homogenization. Since, it was first thought that a crystalline re-arrangement, i.e. the presence of a V-form fraction, was necessary [see the X-ray results obtained for SA,G-2.7 in Example 3b], a sufficient amount of ethanol was added to attain that goal. Then, a volume of ethanol was added after heating the HASCA suspension. Note that the final EtOH/HASCA ratio of 3.2 was chosen to limit ethanol use as much as possible in the process for economical, environmental and safety reasons, while still allowing easy SD. The second step of the process consisted of recovering the product in the form of a dry powder by SD. Traditional chemical dehydration by non-solvent addition was discarded to avoid the necessity of large volumes of organic solvent.

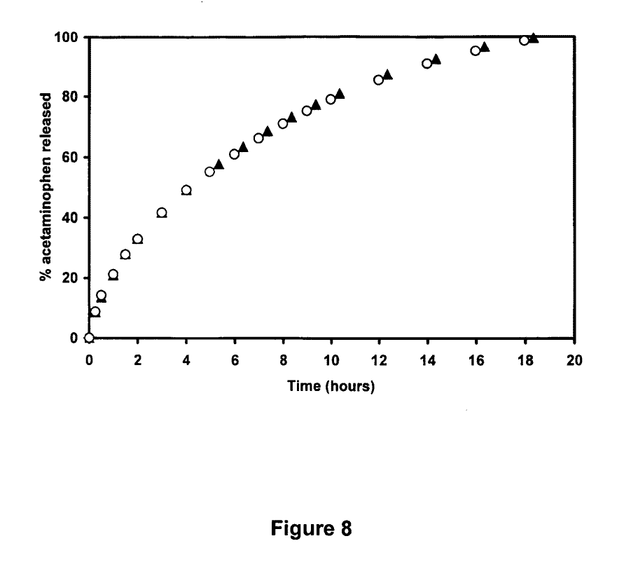

During the first manufacturing step, i.e. heating of the initial hydro-alcoholic suspension, powder and water concentrations are key parameters for the acquisition of excellent binding properties. A compromise must be reached between targeting very high hardness through high-water concentration and limiting viscosity through higher alcohol concentration. In the second step, the optional addition of ethanol before SD is more concerned with decreasing viscosity to easily process the suspension through the spray dryer than having an effect on material properties. Binding properties do not appear to be linked to the presence of a Vh crystal form of amylose, as the most crystalline samples [see Example 3b] are the ones giving the weakest tablets [see Table 4]. On the other hand, high-water concentration leads to high tablet hardness, i.e. inverse conditions leading to the appearance of a Vh form (pseudo V-form) of amylose [see Example 3b and Table 1]. The inventors hypothesized that increasing tablet hardness is possible by first decreasing the particle size of amorphous pregelatinized HASCA through SD. Second, the combination of water and ethanol may have a plasticizer effect, helping partial melting of the excipient and particle re-arrangement under compression. Finally, it appears that variations in hydro-alcoholic composition affect only tableting properties, and surprisingly do not influence the drug-release rate [see Examples 3b and 8c, and FIG. 8]. This is certainly an advantage, making the method robust and focusing on the experimental conditions of heating HASCA hydro-alcoholic suspensions, to optimize tablet strength in the design of an industrial manufacturing process.

Thus, heating amorphous pregelatinized HASCA in a hydro-alcoholic solution, optionally adding then a supplementary volume of a hydro-alcoholic solution to the former, and then spray drying the resulting dispersion, allows quickly obtaining large quantities of spray-dried HASCA suitable for sustained drug-release. Also, this process decreases considerably the required ethanol amounts compared to the former laboratory process, i.e. dispersion in pure water, heating, addition of increasing amounts of ethanol followed by filtration.

To further assess the utility of spray-dried HASCA as a directly-compressible excipient for controlled drug-release, the effects of formulation parameters like compression force (CF), tablet weight (TW), drug-loading and electrolyte particle size on drug-release from HASCA-based matrix systems were also investigated.

### EXAMPLES

### Example 1

### Materials

The following materials were employed in Examples 2 to 15. Amorphous pregelatinized HASCA was obtained in powder form from Roquette Frères (Lestrem, France) and contained approximately 70% of amylose chains and 30% of amylopectin. The DS was equal to 0.045 (number of moles of substituent/number of moles of anhydroglucose). Anhydrous ethanol was purchased from Commercial Alcohol Inc. (Brampton, Ontario, Canada). SA,G-2.7 was obtained exactly like described in U.S. Pat. No. 5,879,707 [see the same patent for the nomenclature and its description]. Acetaminophen was procured from Laboratoires Denis Giroux inc. (Ste-Hyacinthe, Quebec, Canada), and sodium chloride (NaCl) (crystals, lab grade) from Anachemia Ltd. (Montreal, Quebec, Canada). All chemicals were of reagent grade and were used without further purification.

### Example 2

### SD HASCA Manufacturing Process

Suspensions consisting of amorphous HASCA of various weights and 80 g of a hydro-alcoholic solution (containing various % w/w water/ethanol) were heated at 70° C. The solutions were kept at this temperature for 1 hour under stirring. The solution was then cooled down to 35° C. with stirring. A volume of pure ethanol, corresponding to a final alcohol to starch ratio of 4 (ml) to 1 (g), was added “slowly and gradually” to the solution. The final suspension was passed through a Büchi B-1 90 Mini Spray Dryer™ (Büchi, Flawill, Switzerland) at 140° C. to obtain HASCA in the form of a fine, dry powder. The spray-dryer airflow rate was 601 NormLitre/hour.

Table 1 a & b describes the composition of the HASCA suspensions during the two operational steps, i.e. heating of the initial hydro-alcoholic suspensions and SD of the final suspensions: where % w/w WATER=the percent of water by weight in the starting hydro-alcoholic solution in which the powder is dispersed at the beginning of the process. 80 g of this solution serve to disperse each HASCA powder sample.

SOLUTION weight (g)=weight of the hydro-alcoholic solution employed to disperse each HASCA powder sample.
HASCA weight (g)=weight of the HASCA powder added to the hydro-alcoholic solution.
% w/w HASCA-I=[HASCA weight/(HASCA weight+SOLUTION weight)]*100.
% w/w water-I=[(water weight)/(HASCA weight+SOLUTION weight)]*100.
% w/w EtOH-I=[(ethanol weight)/(HASCA weight+SOLUTION weight)]*100.
EtOH added (g)=quantity (g) of ethanol added to the hydro-alcoholic suspension to obtain a SD suspension having a EtOH/HASCA-II ratio of 3.2.
EtOH/HASCA-II=3.2=ratio of the total weight of ethanol on the weight of HASCA in the suspension to be spray-dried.
% w/w HASCA-II=[HASCA weight/(HASCA weight+SOLUTION weight+EtOH added)]*100.
% w/w water-II=[water weight/(HASCA weight+SOLUTION weight+EtOH added)]*100.
% w/w EtOH-II=[EtOH total weight/(HASCA weight+SOLUTION weight+EtOH added)]*100.

TABLE 1
Compositions of (a) HASCA initial hydro-alcoholic suspensions
(heating step) and (b) spray-drying suspensions (drying step)
(a) Initial hydro-alcoholic suspensions

| | | | HASCA | | | |
|---|---|---|---|---|---|---|
| | % w/w | SOLUTION | weight | % w/w | % w/w | % w/w |
| Batch | WATER | weight (g) | (g) | HASCA-I | water-I | EtOH-I |
| A | 65.22 | 80 | 16 | 16.67 | 54.35 | 28.99 |
| B | 65.22 | 80 | 12 | 13.04 | 56.71 | 30.25 |
| C | 65.22 | 80 | 10 | 11.11 | 57.97 | 30.92 |
| D | 74.47 | 80 | 12 | 13.04 | 64.75 | 22.20 |
| E | 74.47 | 80 | 10 | 11.11 | 66.19 | 22.70 |
| F | 83.33 | 80 | 10 | 11.11 | 74.07 | 14.81 |
| G | 100.00 | 80 | 10 | 11.11 | 88.89 | 0.00 |

(b) Spray-drying suspensions

| | EtOH | % w/w | % w/w | % w/w | EtOH/ |
|---|---|---|---|---|---|
| Batch | added (g | HASCA-II | water-II | EtOH-II | HASCA-II |
| A | 23.36 | 13.40 | 43.71 | 42.88 | 3.2 |
| B | 10.56 | 11.70 | 50.87 | 37.43 | 3.2 |
| C | 4.16 | 10.62 | 55.41 | 33.97 | 3.2 |
| D | 18.00 | 10.91 | 54.12 | 34.97 | 3.2 |
| E | 11.60 | 9.84 | 58.60 | 31.56 | 3.2 |
| F | 18.64 | 9.21 | 61.36 | 29.43 | 3.2 |
| G | 32.00 | 8.20 | 65.57 | 26.23 | 3.2 |

All suspensions were subjected to SD.

### Example 3a

### X-ray Diffraction: Method

X-ray diffraction (XRD) was performed to characterize the crystalline or amorphous state of SD HASCA powder samples obtained as described in Example 2. Powder XRD patterns were obtained with an automatic Philips Diffractometer controlled by an IBM PC (50 acquisitions, 3-25° (, 1,100 points; acquisition delay 500 ms), using a Cu anticathode (K(1 1.5405 Å) with a nickel filter. A smoothing function was applied on the spectra for better reading of the peaks. SA,G-2.7 powder was also characterized in the same way.

### Example 3b

### X-ray Diffraction: Results

From the presence of large peaks at 150 and 23.2° (2 ( ) corresponding to d=6.5 and 4.4 (Å), it was concluded that SA,G-2.7 had an essentially amorphous character with a minor crystalline fraction (data not shown). The same was true with lab scale HASCA (data not shown). The crystalline part of SA,G-2.7 was considered as being essentially a V polymorph of amylose. This polymorph did not occur frequently in cereal starch compared to other crystalline forms of starch, i.e. A and B polymorphs [Buléon, A. et al., Single crystals of amylose complexed with a low degree of polymerization, Carbohyd. Polym. 1984, 4 (3), 161-173]. V-amylose, a generic term for crystalline amylose obtained as single helices, co-crystallizes with compounds such as iodine, fatty acids and alcohols [Rundle, R. E. et al., The configuration of starch in the starch-iodine complex. IV. An X-ray diffraction investigation of butanol-precipitated amylose, J. Am. Chem. Soc. 1943, 65, 2200-2203; Godet, M. C. et al., Structural features of fatty acid-amylose complexes, Carbohyd. Polym, 1993, 21 (2-3), 91-95; Hinkle, M. E. et al., X-ray diffraction of oriented amylose fibers. III. The structure of amylose-n-Butanol complexes, Biopolymers 1968, 6, 1119-1128; Buléon, A. et al., Single crystals of amylose complexed with isopropanol and acetone, Int. J. Biol. Macromol. 1990, 12 (1), 25-33]. Especially for alcohols, these types of complexes mainly occur by precipitation of amylose with alcohols (methanol, ethanol, n-propanol) in heated, aqueous solution [Valletta, R. M. et al., Amylose “V” complexes: low molecular weight primary alcohols, J. Polym. Sci.: Part A 1964, 2, 1085-1094; Bear, R. S., The significance of the V X-ray diffraction patterns of starches, J. Am. Chem. Soc. 1942, 64, 1388-1391; Helbert, W. et al., Single crystals of V amylose complexed with n-butanol or n-pentanol: structural features and properties, Int. J. Biol. Macromol. 1994, 16 (4), 207-213; Katz, J. R. et al., IX Das Röntgenspektrum der α-Diamylose stimmt weitgehend mit dem gewisser Stärkepräparate überein, Z. Physik. Chem. 1932, A158, 337]. This might explain the presence of amylose-acetone or amylose-ethanol complexes in SA,G-2.7 or HASCA produced according to the original lab-scale process.

On the other hand, pilot-scale HASCA displays the characteristic pattern of a amorphous powder (data not shown), and is industrially produced as such for economical and technical reasons.

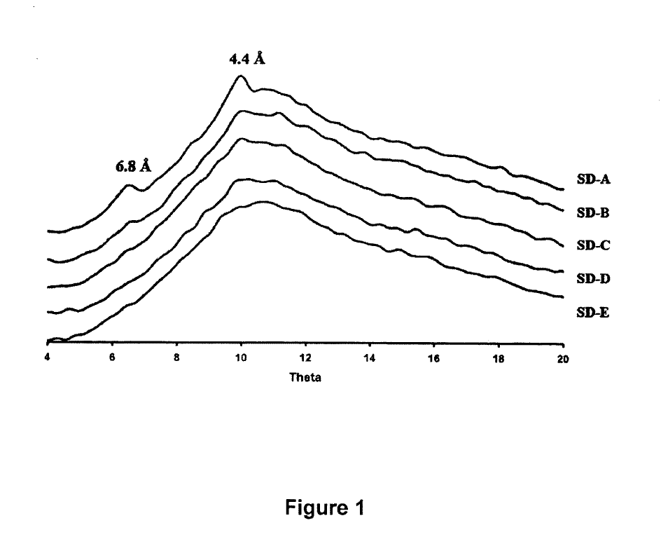

The XRD results on typical SD HASCA samples obtained as described in Example 2 appear in FIG. 1. The presence of a V-type complex in HASCA spray-dried batches was verified by XRD. The XRD diagram of the SD-A sample reveals reflections at Bragg angles 2θ=6.80°, 12.96°, 19.92°, and a less intense one at 2θ=21.88°. This XRD pattern is close to those reported previously for pure amylose-ethanol complexes [Bear, R. S., The significance of the V X-ray diffraction patterns of starches, J. Am. Chem. Soc. 1942, 64, 1388-1391]. Table 2 reports that such peaks are, in fact, more characteristic of the Vh amylose polymorph although the diffraction peaks are broader [Le Bail, P. et al., Polymorphic transitions of amylose-ethanol crystalline complexes induced by moisture exchanges, Starch/Stärke 1995, 47 (6), 229-232]. A Vh amylose structure, often called a pseudo V-form, is indeed characterized by a larger structure. The V-type helix is a form of order existing in both crystalline and amorphous regions [Veregin, R. P. et al., Investigation of the crystalline “V” amylose complexes by high-resolution carbon-13 CP/MAS NMR spectroscopy, Macromolecules 1987, 20 (12), 3007-3012].

TABLE 2
Observed distances (Å) for HASCA and different types of V-
amylose complexes reported in the literature

| | Organic | | | | |
|---|---|---|---|---|---|
| Reference | solvent | Observed d-spacings (Å) | | | |
| 7SD-HASCA | ethanol | 4 | 4.4 | 6.8 | 12.9 |
| This work | | | | | |
| Pure V-amylose | ethanol | | 4.5 | 7 | |
| Bear (1942) supra | | | | | |
| Pure Vh amylose | ethanol | 3.93 | 4.47 | 6.84 | 11.87 |
| Le Bail et al. (1995) supra | | | | | |

A progressive loss of the crystalline part is observed when decreasing % w/w HASCA-I and/or increasing % w/w water-I in the different spray-dried suspensions (Table 1 and FIG. 1). In fact, usually higher volumes of ethanol are required to obtain highly crystalline complexes. Here, the crystalline part becomes more and more diluted compared to the amorphous part to a point that it is no longer detectable by XRD. Note that SD-F and SD-G are not differentiable from SD-E and are not presented in the figure for the purpose of clarity. SD samples generate the same type of patterns, and thus the same type of structures, i.e. a pseudo V-form dispersed in an amorphous matrix, although their respective proportions cannot be determined exactly here, until of course the pseudo V-form can no more be detected.

### Example 4a

### Scanning Electron Microscopy (SEM): Method

The morphology of the samples prepared according to Example 2 was studied by SEM (Hitachi S 4000, Hitachi, Japan). Prior to investigation, the samples were mounted on double adhesive tape and sputtered with a thin gold palladium coat.

### Example 4b

### Scanning Electron Microscopy (SEM): Results

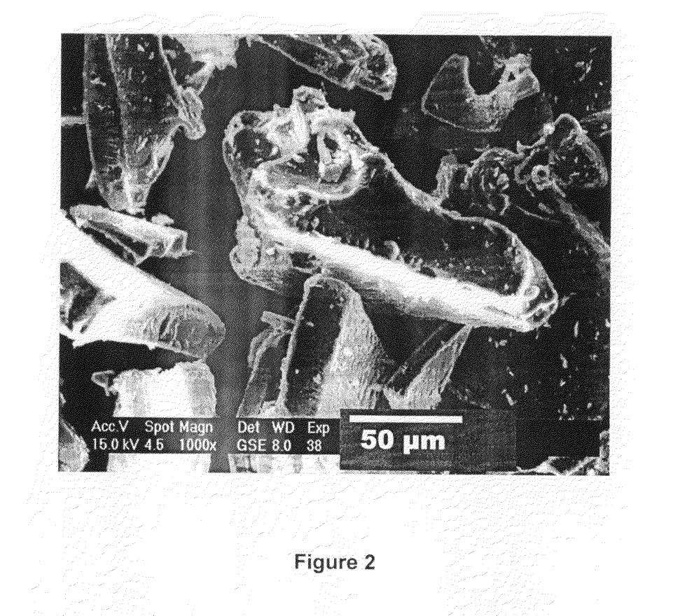

A SEM picture of the starting material, i.e. amorphous HASCA obtained at the pilot level, appears in FIG. 2. The initial product consisted of large, flat and splinter-shaped particles.

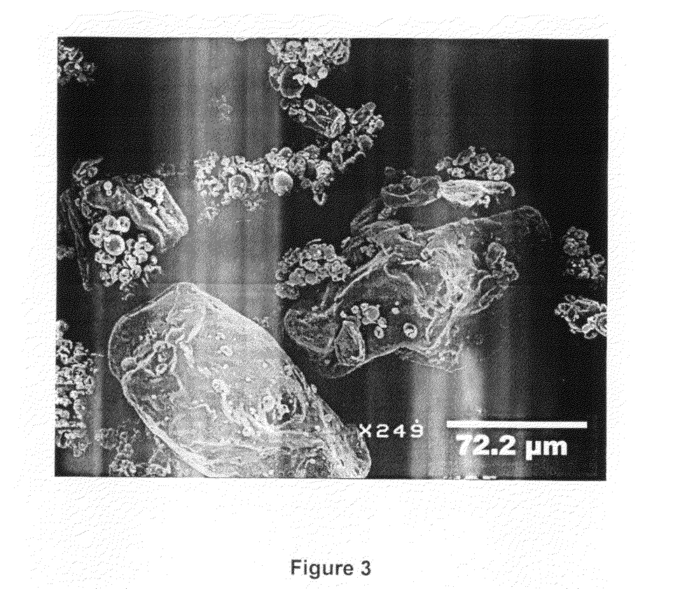

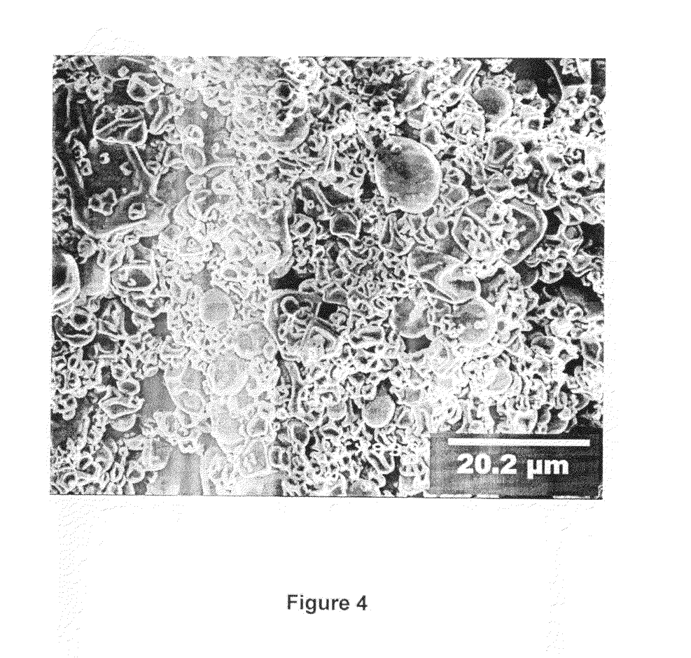

Products obtained by SD were also characterized by SEM. Samples from spray-dried suspensions were characterized by more or less collapsed spherical particles of various sizes (FIGS. 3 and 4). This typical shape appears when, under the drying action, the solid forms a crust around each droplet, raising vapour pressure inside. Collapsed particles are created when the vapour is released. SD-A (FIG. 3) contains large, smooth, polyhedral particles with small more or less collapsed spherical particles often agglomerated on them. On the other hand, SD-D is composed of small collapsed spherical particles together forming larger agglomerates (FIG. 4). The main preparation difference between these two samples is, on the one hand, the higher % w/w HASCA-I for SD-A, and on the other hand, the lower % w/w water-I for SD-A compared to SD-D (Table 1). Both factors do not favour HASCA's complete dissolution for SD-A compared to SD-D. In fact, the water/ethanol (p/p) ratio is approximately equal to 1.9 for SD-A and 2.9 for SD-D. This could explain the presence of these large particles in SD-A, most probably corresponding to the initial amorphous particles that are only partially dissolved. Thus, in the case of SD-D, a major part of the initial starch product is dissolved before being spray-dried, and the general appearance will be more typical of a spray-dried product. On the one hand, increasing water concentration helps to dissolve HASCA, which is a necessary condition for the formation of a pseudo-V-amylose complex, because amylose chains have to be free for that purpose. On the other hand, the SD process being developed to decrease ethanol concentration will not lead to amounts of pseudo-V-amylose detectable by XRD, even if large amounts of amylose are dissolved previously (FIG. 1).

### Example 5a

### True Density: Method

Helium pycnometry (Multivolume pycnometer 1305™, Micromeritics, Norcross, Ga., USA) was undertaken. Sample holder volume was 5 ml, and HASCA sample weight was between 0.5 and 1.5 g. The results are expressed in g/cm³.

### Example 5b

### True Density: Results

The true density values of samples SD-A and SD-D (see Example 2 for their preparation) are enumerated in Table 3.

TABLE 3
Density values of typical HASCA samples.

| | Density |
|---|---|
| HASCA type | (g/cm³) |
| SD-A | 1.26 ± 0.03 |
| SD-D | 1.04 ± 0.10 |
| Amorphous starting material | 1.48 ± 0.01 |

True density results may be interpreted in light of the information garnered by SEM. SD-D had a lower true density than SD-A. Indeed, SD-D was composed of small, more or less collapsed spherical particles resulting from the SD of HASCA, which had almost been fully dissolved (FIG. 4). It has been mentioned earlier that under the drying action, the solid in the solution formed a crust around each droplet, raising vapour pressure inside. Eventually, collapsed particles were formed when the vapour was released. Such structures were obviously less dense than plain particles. Indeed, SD-A contained large, smooth, polyhedral particles with small, more or less collapsed spherical particles often agglomerated on them (FIG. 3). These large particles appeared as plain particles and likely did not present porous structures, which resulted in increased global true density. Also, SD-A had a lower true density than amorphous particles. Again, this could have been related to the bulk aspect of small particles. Due to surface coagulation and vapour release, SD-A small particles may have become closed structures with internal porosity unlike that of amorphous particles. In fact, amorphous HASCA had a much higher density than all spray-dried samples, which confirms our interpretation of the true density values based on the open or closed porosity of HASCA particles.

### Example 6a

### Surface Area: Method

Krypton adsorption/desorption isotherms were measured with a Micromeritics ASAP 2010TM instrument (Micromeritics, Norcross, Ga., USA). HASCA samples were outgassed overnight at 200° C. Specific surface area was calculated from adsorption data in the relative pressure range of 0.10 to 0.28, included in the validity domain of the Brunauer-Emmett-Teller (BET) equation. BET-specific surface area was calculated from the cross-sectional area of 0.218 nm² per krypton molecule, following I.U.P.A.C. recommendations.

### Example 6b

### Surface Area: Results

The specific surface area value of a typical SD sample prepared as described in Example 2, i.e. SD-D, has been obtained to gain supplementary information on the type of product obtained by SD (S=2.28 m2/g).

### Example 7a

### Tablet Hardness: Method

SD HASCA tablets weighing 200 mg were prepared by direct compression. The excipient, obtained as described in Example 2, was compressed in a hydraulic press (Workshop Press PRM 8TM type, Rassant Industries, Chartres, France) at a compaction load of 2.5 tons/cm² with a dwell time of 30 s (flat-faced punch die set). The diameter of all the tablets was 12.6 mm. Tablet hardness (Strong-Cobs or SC) was quantified with a hardness tester (ERWEKA® Type TBH 200, Erweka Gmbh, Heusenstamm, Germany). The data presented here are the mean values of three measurements.

### Example 7b

### Tablet Hardness: Results

It was not possible to obtain tablets with the initial amorphous pregelatinized HASCA pilot batch, even at very high compression forces (up to 5 tons/cm2). Table 4 gives the hardness values of compacts generated by SD HASCA obtained as described in Example 2. Clearly, the SD process produces tablets whose mechanical properties vary from adequate to excellent.

TABLE 4
Hardness determined for 200-mg tablets (Ø = 12.6 mm,
F = 2.5 tons/cm²) of pure SD HASCA

| | Mean ± SD |
|---|---|
| HASCA type | (Strong-Cobbs) |
| SD-A | 8.5 ± 0.4 |
| SD-B | 15.3 ± 0.4 |
| SD-C | 20.2 ± 0.1 |
| SD-D | 20.4 ± 1.3 |
| SD-E | 24.3 ± 1.2 |
| SD-F | 26.0 ± 0.2 |
| SD-G | 26.6 ± 0.2 |

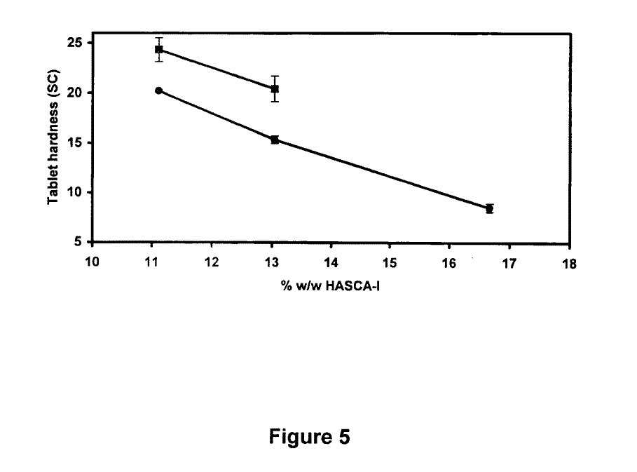

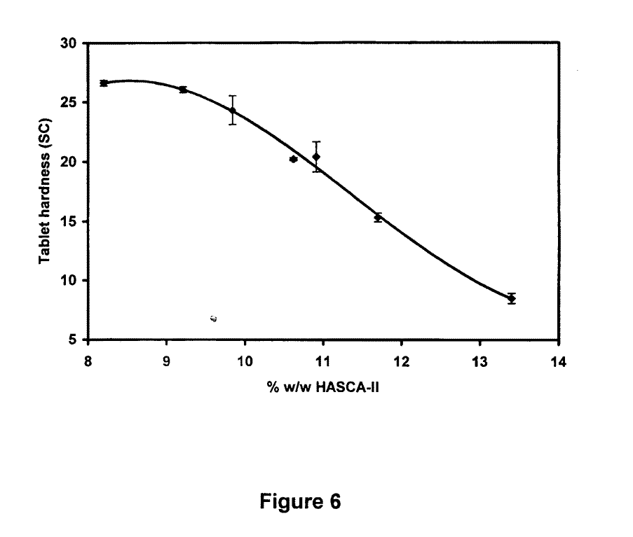

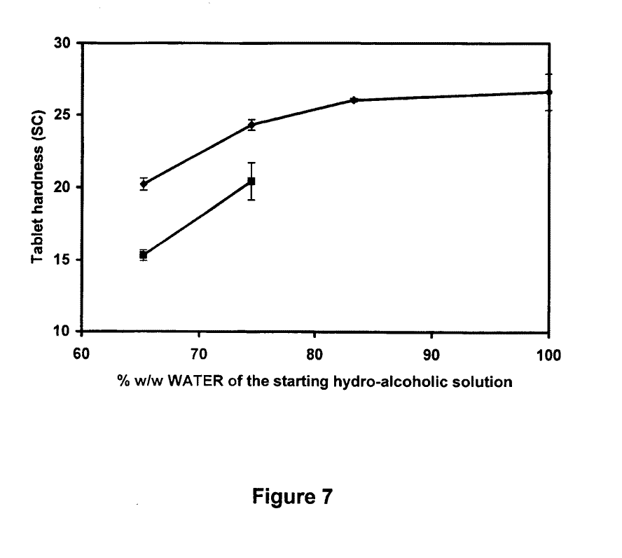

Some general trends can be underlined concerning the concentration of the different compounds in the initial hydro-alcoholic suspension and the SD suspension. FIGS. 5-7 depict the influence of various parameters of the initial hydro-alcoholic and SD suspensions on tablet hardness. FIG. 5 charts the influence of % w/w HASCA-I of the initial hydro-alcoholic HASCA suspensions on HASCA tablet strength for different water concentrations. A quasi-linear relationship was observed between tablet hardness and % w/w HASCA-I of the initial hydro-alcoholic solution for the 11-17% w/w range. Interestingly, lower water concentrations of the starting hydro-alcoholic solution followed the same trend in parallel but gave higher tablet hardness values. We can assume that decreasing powder weight while keeping the same water concentration allowed better dissolution of the initial HASCA dispersion. Considering that the initial HASCA particles did not show any binding properties, we may emit the hypothesis that the newly-formed small particles are responsible for the increased hardness. Indeed, we can suppose that augmenting the number of smaller particles enlarged the surface area of the particulate product and, consequently, provided a higher number of binding points. The progressive disappearance of the large HASCA particles, due to their progressive dissolution induced by the rising water/HASCA ratio, thus elicited increased hardness. FIG. 6 profiles the influence of HASCA concentration in the SD dispersion (% w/w HASCA-II) on tablet strength. The final ethanol addition, which allowed apparent viscosity reduction of the suspension before SD, did not really change the earlier observations. Surprisingly, the relationship appeared to be sigmoid when values obtained for the different water concentrations were pooled, and a maximum hardness value was obtained near 9.5% p/p with less HASCA. FIG. 7 enunciates the influence of % w/w WATER of the starting hydro-alcoholic solution on tablet strength for different weights of HASCA powder dispersed in 80 g of the hydro-alcoholic solution. Clearly, increasing water concentration in the starting hydro-alcoholic solution for the same powder quantity enhanced tablet hardness until a certain limit was reached.

Further, an aqueous HASCA solution was prepared under the same conditions as for SD-G, but no ethanol was added before SD. Not only was this solution difficult to manipulate because of its high viscosity, but it was also impossible to end the experiment with a lab-scale spray dryer. The high viscosity of this solution seemed to attract too many problems, confirming the necessity of the hydro-alcoholic solution in the case of industrial manufacturing.

Thus, the two key parameters for HASCA excellent binding properties are powder and water concentrations during the first manufacturing step, i.e. heating of the initial hydro-alcoholic suspension. A compromise must be reached between targeting very high hardness through a high-water concentration and limiting viscosity through higher alcohol concentration. In the second stage, the addition of ethanol is more concerned with decreasing viscosity to easily process the suspension through the spray dryer than having an effect on material properties.

Finally, binding properties do not appear to be linked to the presence of a Vh form of amylose, as the most crystalline samples are the ones giving the weakest tablets (FIG. 1 and Table 4). On the other hand, tablet hardness rose with water concentration, though these conditions did not lead to the appearance of a Vh form of amylose. It can be hypothesized that increasing tablet hardness was obtained by first decreasing the particle size of amorphous HASCA through SD. Second, the combination of water and ethanol may have had a plasticizer effect, helping partial melting of the excipient and particle re-arrangement under compression. The peculiar melting process was demonstrated earlier by SEM and porosimetry in the case of SA,G-2.7, although no explanation was provided [Moghadam, S. H.; Wang, H. W.; Saddar El-Leithy, E.; Chebli, C.; Cartilier, L., Substituted amylose matrices for oral drug delivery. Biomed. Mater. 2007, 2, S71-S77].

### Example 8a

### Drug-Release Evaluation: Tablet Preparation

Matrix tablets were prepared by direct compression. SD HASCA (prepared as described in Example 2), acetaminophen and NaCl were dry-mixed manually in a mortar. 600-mg tablets, containing 40% of acetaminophen as a model drug, 27.5% of NaCl and 32.5% of SD HASCA, were produced to investigate the influence of thermal treatment and SD on the release characteristics of SD HASCA tablets. They were prepared in a hydraulic press (Workshop Press PRM 8 type, Rassant Industries, Chartres, France). All tablets were compressed at 2.5 tons/cm2 for 30 s. The diameter of the tablets was 1.26 cm.

### Example 8b

### Drug-Release Evaluation: Method

The drug-release properties of some typical SD HASCA matrix tablets were assessed by an in vitro dissolution test. Since HASCA is an ionic polymer used for oral, sustained drug-release, in vitro release experiments were conducted in a pH gradient simulating the pH evolution of the gastrointestinal tract. The tablets were placed individually in 900 ml of an hydrochloric acid medium (pH 1.2) simulating gastric pH, at 37° C., in U.S.P. XXIII Dissolution Apparatus No. 2 equipped with a rotating paddle (50 rpm). They were then transferred to a phosphate-buffered medium (pH 6.8) simulating jejunum pH, and finally, transferred to another phosphate-buffered medium (pH 7.4) simulating ileum pH, until the end of the test. The dissolution apparatus and all other experimental conditions remained the same. The pH gradient conditions were: pH 1.2 for 1 hour, pH 6.8 for 3 hours, and pH 7.4 until the end of the dissolution test (24 hours). The amount of acetaminophen released at predetermined time intervals was followed spectrophotometrically (244 nm). All formulations were tested in triplicate. The drug-release results are expressed as cumulative % in function of time (hours).

### Example 8c

### Drug-Release Evaluation: Results

Typical drug-release profiles from matrix tablets made of spray-dried HASCA are shown in FIG. 8. SD-A and SD-D were chosen because they present different crystalline levels and different binding properties. Acetaminophen release was found to be similar for the two samples. The time for 95% drug-release was equal to 16:30 hours, and it could be said that SD HASCA matrix systems exhibited sustained drug-release properties. Thus, combined with the heating of HASCA hydro-alcoholic suspensions, the SD process was able to restore binding and sustained drug-release properties. Further, it appears that within the limits of this protocol, variations in hydro-alcoholic composition only affected tableting properties, and did not influence the drug-release rate. The presence of the Vh form of HASCA appears to be unnecessary to obtain sustained drug-release (FIGS. 1 and 8), but also its concentration does not influence the drug-release process, provided it remains as a minor component in the amorphous matrix. This is certainly an advantage as it makes the method robust and allows us to focus on the experimental conditions of heating HASCA hydro-alcoholic suspensions to optimize tablet strength in the design of an industrial manufacturing process.

### Example 9

### SD HASCA-Manufacturing Process

First, 10 g of amorphous pregelatinized HASCA were dispersed under stirring in 80 grams of a hydro-alcoholic solution (16.66% w/w ethanol) at 70° C. (see Example 1 for the description of materials). The solution was kept at this temperature for 1 hour under stirring. It was then cooled to 35° C. under stirring. A volume of 23.5 ml of pure ethanol was added “slowly and gradually” to the solution. Note that the final alcohol to starch ratio w/w was 3.2 (or 4 ml/g). The final solution was passed through a Büchi B-290 Mini Spray-Dryer™ at 140° C. to obtain HASCA in dry powder form. Spray-dryer airflow was 601 NormLitre/hour and liquid flow was 0.32 litre/hour.

### Example 10

### Tablet Preparation Method

Tablets with a diameter of 1.26 cm were prepared by direct compression, i.e. manual dry-mixing of acetaminophen, SD HASCA (prepared as described in Example 9), and sodium chloride (NaCl) in a mortar, followed by compression in a 30-ton manual pneumatic press (C-30 Research & Industrial Instruments Company, London, U.K.). The exact composition of the tablets is described further in Examples 11b, 12, 13, 14, 15, 16a and 17. Despite poor powder flow properties, no lubricant was added to the formulation because it was unnecessary, considering the peculiar tableting process involved here, i.e. manual pneumatic compression. Furthermore, it was demonstrated earlier that magnesium stearate, at standard levels, did not influence the in vitro release profile of HASCA matrix tablets containing NaCl as well as their integrity [see Cartilier, L. et al., Tablet formulation for sustained drug-release, Canadian Patent Application No. 2,591,806, Dec. 20, 2005].

### Example 11 a

### Tablet Hardness Testing: Method

Tablet hardness was quantified in a PHARMATEST™ type PTB301 hardness tester. These tests were performed on 200-mg SD HASCA (manufactured as described in Example 9) tablets with a diameter of 1.26 cm obtained under a CF of 2.5 tons/cm² in a 30-ton manual pneumatic press (C-30 Research & Industrial Instruments Company, London, U.K.). Typical tablets containing acetaminophen and NaCl (prepared following the method described in Example 10) were also analysed. The results are expressed in Strong-Cobs (SC).

### Example 11b

### Tablet Hardness Testing: Results

A mean hardness value of 27.0±1.5 SC (equivalent to 189 N) was determined from 10 pure 200-mg SD HASCA tablets. For a formulation containing 40% acetaminophen, 27.5% NaCl and 32.5% SD HASCA, the hardness value for 400-mg tablets, compressed at 2.5 tons/cm², was 16.9 SC, and for 600-mg tablets, it was 39.7 SC. Considering that SD HASCA represents only 32.5% of the total powder and that NaCl is known to have poor compaction properties, these results prove the potential of SD HASCA for industrial tableting applications. Another advantage of such good compaction properties is that no binder is required, which simplifies formulation optimization.

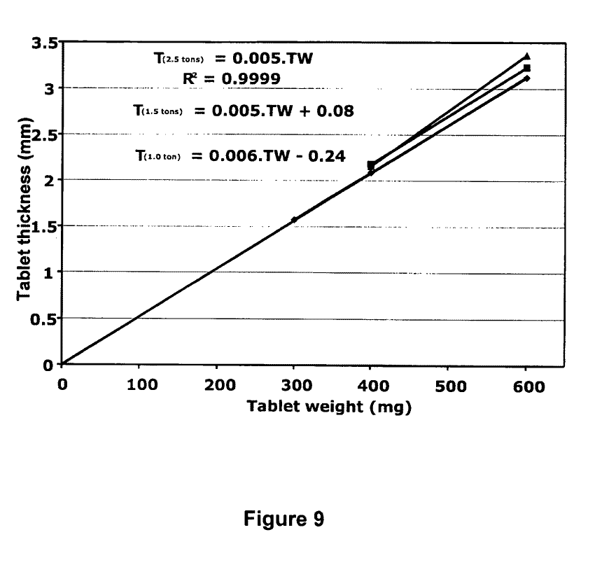

The relationship between tablet weight (TW) and compression force (CF) versus tablet thickness (TT) was investigated to understand the good binding properties of SD HASCA. During tablet preparation, diameter remained the same for each TW, and thus, the only geometric variable, which had to be considered here was TT. These results are presented in Table 5 and FIG. 9, which reveal a perfect linear relationship between TW and TT.

TABLE 5
Influence of compression force (CF) on tablet thickness (TT).

| Formulation (% w/w) | | | TW | CF | TT |
|---|---|---|---|---|---|
| Drug | HASCA | NaCl | (mg) | (t/cm²) | (mm) |
| 40 | 32.5 | 27.5 | 600 | 2.5 | 3.12* |
| 40 | 32.5 | 27.5 | 600 | 1.5 | 3.23 ± 0.03 |
| 40 | 32.5 | 27.5 | 600 | 1.0 | 3.36 ± 0.01 |
| 40 | 32.5 | 27.5 | 400 | 2.5 | 2.09* |
| 40 | 32.5 | 27.5 | 400 | 1.5 | 2.18 ± 0.01 |
| 40 | 32.5 | 27.5 | 400 | 1.0 | 2.16 ± 0.02 |
| 40 | 32.5 | 27.5 | 300 | 2.5 | 1.57 ± 0.01 |

TW, tablet weight
*Tests performed on two samples only

The slope remains almost identical, even for the lowest CF, i.e. 1 ton/cm². Thus, densification was the same for all CFs, meaning that particle re-arrangement was optimal and that some peculiar phenomenon took place, even at low CFs, leading to an intense densification process. This phenomenon was already reported in the case of SA,G-2.7, where a sintering by total or partial melting process was seen, which also confirmed the excellent binding properties recorded previously for SA,G-n tablets. On the other hand, Table 5 indicates that, practically, CF does not influence TT. A very slight effect of CF on TT was apparent only in the case of 600-mg tablets, i.e. a 7% decrease in TT corresponded to a CF increase from 1 to 2.5 tons. Note that the tablets did not contain any lubricant. In these conditions, CF was probably not sufficient to allow maximal densification. Indeed, it has already been observed that the addition of a lubricant to SA,G-2.7 fully removes the slight influence of CF on TT, even for larger TWs [see Wang, H. W., Développement et évaluation de comprimés enrobés à sec, à base d'amylose substitué, Mémoire M. Sc., Faculté de pharmacie, Université de Montréal, August 2006].

### Example 12

### Drug-Release Evaluation: Effect of CF on the Dissolution Rate

Tablets containing 40% of acetaminophen as model drug, 27.5% of NaCl and 32.5% of SD HASCA (manufactured as described in Example 9) were prepared as described in Example 10 to study the effects of CF on the dissolution rate. They weighed 400 or 600 mg each and were subjected to various CFs: 1, 1.5 and 2.5 tons/cm² for 30 s. The drug-release properties of the SD HASCA matrix tablets were assessed by the in vitro dissolution test already described in Example 8b. Drug-release profile reproducibility was excellent as the standard-deviation values observed for the % of drug released versus time were generally lower than 1%, ranging from 0.2 to 2.4% for experiments described in Examples 12 to 15. Standard-deviation bars were omitted in the figures for clarity.

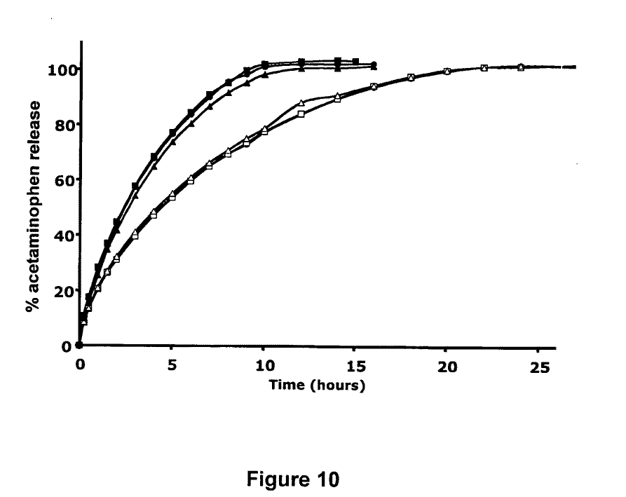

FIG. 10 charts the effect of CF on the acetaminophen release profile of 600- and 400-mg HASCA matrix tablets. Between 1 and 2.5 tons/cm², CF does not significantly influence drug-release from HASCA matrices. This range of CFs has been selected because it covers the normal range of compaction forces employed at the industrial level. The slight increase in the drug-release rate for 400-mg tablets at low CFs, i.e. 1 and 1.5 tons/cm², could be explained by the fact that 400-mg swollen matrices are very thin and subject to slight erosion due to tablet movement on the grid in the dissolution tester. Erosion was not apparent for 600-mg tablets.

SD HASCA matrices have some specific features regarding the influence of CF on water and drug-transport mechanisms. SD HASCA matrices do not show any importance of CF on the amplitude of the burst effect, on the time-lag, or on the drug-release rate. On the other hand, the gelation properties and drug-release rate of some typical hydrophilic matrices, such as higher plant hydrocolloidal matrices, are drastically affected by changes in compression [Kuhrts, E. H., U.S. Pat. No. 5,096,714; Ingani H. and Moës A., Utilisation de la gomme xanthane dans la formulation des matrices hydrophiles, Proceedings of the 4^{th} International Conference on Pharmaceutical Technology, APGI, Paris, June 1986, pp 272-281]. Furthermore, it has been reported that in a number of cases, CF had no or very little influence on the drug-release rate from HPMC hydrophilic matrix tablets, at least beyond a certain CF level [Varma, M. V. S. et al., Factors affecting the mechanism and kinetics of drug release from matrix-based oral controlled drug delivery systems, Am. J. Drug Deliv., 2(1), 43-57 (2004); Ford, J. L. et al., Importance of drug type, tablet shape and added diluents on release kinetics from hydroxypropyl methylcellulose matrix tablets, Int. J. Pharm., 40, 233-234 (1987); Velasco, M. V. et al., Influence of drug: hydroxypropylmethylcellulose ratio, drug and polymer particle size and compression force on the release of diclofenac sodium from HPMC tablets, J. Contr. Rel., 57, 75-85 (1999)], whereas in other cases, CF had an effect on this parameter [Levina, M., Influence of fillers, compression force, film coatings and storage conditions on performance of hypromellose matrices, Drug Deliv. Technol., 4(1), January/February, Excipient update, (2004)] or only on the time-lag before the establishment of quasi-stationary diffusion [Salomon, J. -L. et al., Influence de la force de compression, de la granulométrie du traceur et de l'épaisseur du comprimé, Pharm. Acta Helv., 54(3), 86-89 (1979)].

The independence of the drug-release profile from CF is a very interesting feature of SD HASCA as it facilitates its industrial applications and one does not need to pay attention to the usual slight variations in CF that occur during industrial manufacturing.

### Example 13

### Drug-Release Evaluation: Effect of TW on the Dissolution Rate

Tablets containing 40% of acetaminophen, 27.5% of NaCl and 32.5% of SD HASCA (manufactured as described in Example 9) were also produced as described in Example 10 to investigate the influence of TW on the dissolution rate. They weighed 300, 400 or 600 mg and were all compressed at 2.5 tons/cm² for 30 s. The drug-release properties of the SD HASCA matrix tablets were assessed by the in vitro dissolution test already described in Example 8b.

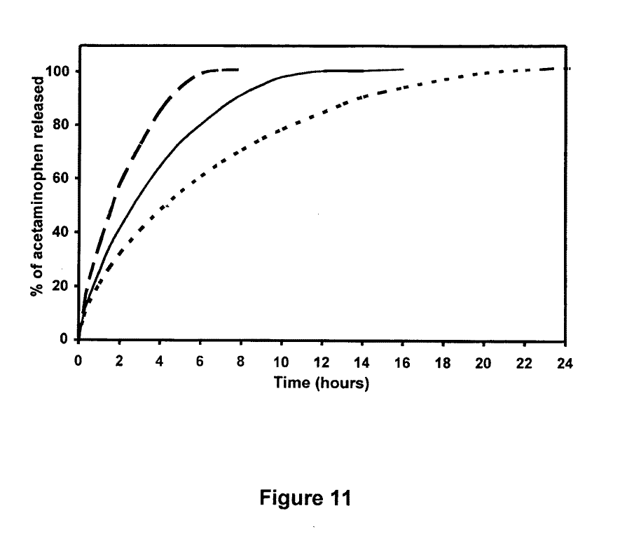

The influence of TW on the drug-release profile from SD HASCA matrices is depicted in FIG. 11. Total drug-release time increased as TW rose. Once-a-day, sustained drug-release dosage forms were easily obtained with SD HASCA technology.

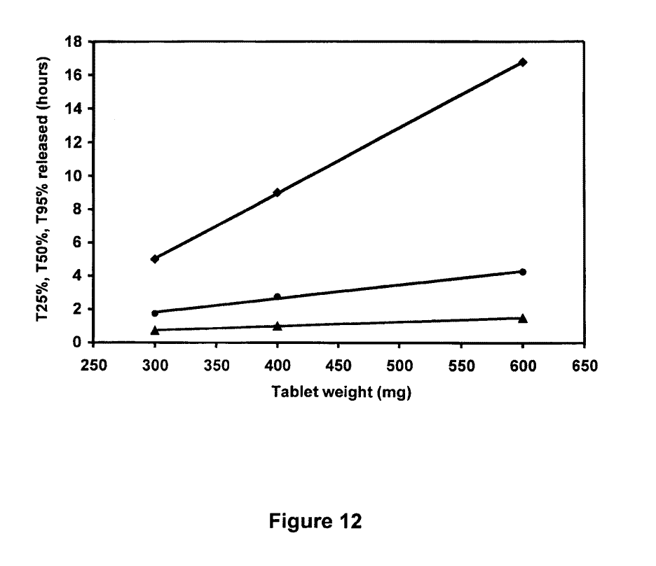

The strong dependence of drug-release on TW is further confirmed in FIG. 12. The time for 25% of drug-release (T25%) is considerably less affected by TW variation than the time for 95% of drug-release. This T25% time value relates to the burst effect, and thus depends on the amount of drug at the tablet surface available for immediate dissolution and release in the medium. Further, in theory, when doubling TW, one doubles tablet height and drug content, with the % drug being kept constant, but increases the total surface by only 25%; in practice, the increase in surface was around 20% in the present case (for example, the external surface of a 600-mg tablet was only 1.2 times the surface of a 300-mg tablet, 3.72 cm² and 3.11 cm², respectively). However, the time for 95% of release increases 3.4 times, showing that a non-linear relationship exists between surface and release-time. In contrast, it is striking that a linear relationship has been observed between TW and release time. After the burst period, a gel layer is formed around the dry core, hindering inward water penetration and outward drug diffusion. Consequently, drug-release is controlled by its diffusion through the gel layer. One may consider that the surface, thickness and structure of the gel layer are nearly the same for each TW, as the eluting medium penetrates at the same rate to a certain depth of the tablet, regardless of its size, where hydration, polymer relaxation, and molecular rearrangement occur, allowing gel-formation [Varma, M. V. S. et al., Factors affecting the mechanism and kinetics of drug release from matrix-based oral controlled drug delivery systems, Am. J. Drug Deliv., 2(1), 43-57 (2004)]. However, the dry and/or partially hydrated core increases in function of TW. This core may be viewed as a drug reservoir. Thus, more time will be required to empty it, and it will be proportional to the concentration of the internal reservoir, and, hence, proportional to TW, which is reflected by the linear relationship exhibited by T95%, T50% and T25%.

### Example 14

### Drug-Release Evaluation: Effect of Drug-Loading on the Dissolution Rate

Tablets containing 10 or 40% of acetaminophen as model drug, 27.5% of NaCl and SD HASCA (manufactured as described in Example 9) were prepared as described in Example 10 to study the effects of drug-loading on the dissolution rate. They weighed 600 mg each and were subjected to a CF of 2.5 tons/cm² for 30 s. The drug-release properties of the SD HASCA matrix tablets were assessed by the in vitro dissolution test already described in Example 8b.

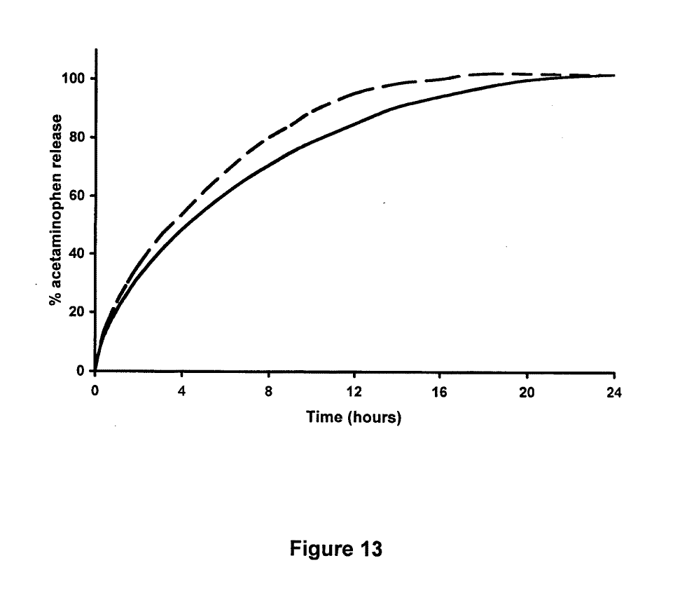

FIG. 13 reports on the influence of drug-loading on acetaminophen release from 600-mg HASCA tablets compressed at 2.5 tons/cm² containing 10% or 40% acetaminophen. An increase in drug-loading corresponded to an increase in total release time (17 hours for 10% loading compared to 23 h for 40% loading). Usually, the opposite observation is made with hydrophilic matrices. It should be noted that despite small cracks appearing gradually on the tablet surface since the 7^{th} hour (see Example 16b), no burst could be detected on the drug-release profile of tablet formulations containing 10% of acetaminophen (FIG. 13). We hypothesize that HASCA matrix tablets, after crack formation and exposure of new surfaces to the external medium [see Cartilier, L. et al., Tablet formulation for sustained drug-release, Canadian Patent Application No. 2,591,806, Dec. 20, 2005], will rapidly form a tight cohesive gel able to maintain control on drug-release. In a certain way, it is as if the gel layer controlling drug-release is able to “heal”, thus protecting the internal drug reservoir, though the dosage form manufacturing process generates a matrix without any doubt. Also, if we suppose that a peculiar gel layer forms around a dry and partially gelified core, we may consider that increasing matrix drug-loading raises the drug concentration in a core of approximately the same size, and that longer time will be needed to drain this higher drug quantity out of the swollen matrix.

Nevertheless, the present work confirms that SD HASCA matrices have a good capacity to control drug-release for high concentrations of a soluble drug like acetaminophen.

### Example 15

### Drug-Release Evaluation: Effect of NaCl Particle Size on the Dissolution Rate

NaCl, a model electrolyte, was added to the tablet formulation to maintain the integrity of HASCA swollen matrices [Cartilier, L. et al., Tablet formulation for sustained drug-release, Canadian Patent Application No. 2,591,806, Dec. 20, 2005]. NaCl being an important component in the formulation of HASCA matrix tablets, it is interesting to evaluate the role of NaCl particle size in the release rate of a typical formulation. 600-mg SD HASCA tablets containing 40% of drug and 27.5% of NaCl were prepared in the same conditions as described as in Examples 9 and 10 to examine the impact of NaCl particle size on the drug-dissolution rate. The various granulometric fractions tested in these experiments were: 600-125 microns (the usual particle size distribution used for all other experiments in the present work), 600-425 microns, and 300-250 microns. The drug-release properties of the SD HASCA matrix tablets were assessed by the in vitro dissolution test already described in Example 8b.

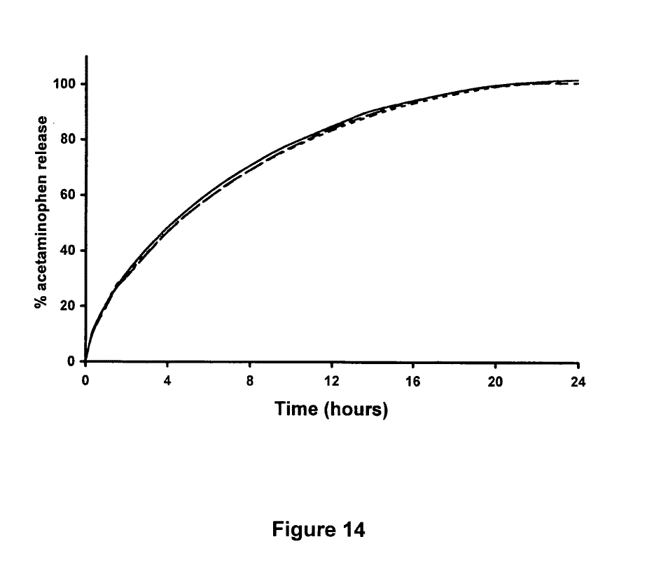

FIG. 14 displays the absence of effect of NaCl particle size on the acetaminophen-release profile from 600-mg tablets containing 40% acetaminophen and 27.5% NaCl, which is a further advantage of such tablets.

### Example 16a

### Evaluation of Swollen Tablet Integrity: Method

It has been reported previously that HASCA matrix tablets crack and separate into two parts loosely attached at their centre, or even split into several parts when swollen in aqueous solution, particularly when going through a pH gradient. The addition of an electrolyte provided complete stabilization of the swollen matrix structure or at least significantly delayed the appearance of the above-mentioned problems and/or decreased their intensity [see Cartilier, L. et al., Tablet formulation for sustained drug-release, Canadian Patent Application No. 2,591,806, Dec. 20, 2005]. Thus, a standardized method was designed to describe the modifications occurring during tablet immersion in aqueous solutions.

SD HASCA matrix tablets, similar to the ones tested for drug-release (see Table 6), were placed individually in 900 ml of an hydrochloric acid solution (pH=1.2), at 37° C., in the U.S.P. XXIII Dissolution Apparatus No. 2 with rotating paddle (50 rpm). After remaining in the acidic solution for 1 hour, the tablets were transferred for 3 hours to a phosphate-buffered solution (pH=6.8), at 37° C., in the same U.S.P. XXIII Dissolution Apparatus No. 2 equipped with rotating paddle, then to a phosphate-buffered solution (pH=7.4) under similar conditions until the end of the test. To prevent the tablets from sticking to the glassware, a small, curved grid was placed at the bottom of the recipient so that drug-release could occur from all sides of the matrix. All formulations were tested in triplicate.

The observation of macroscopic transformations was standardized in a table with specific qualitative terms describing them and recording the moment at which they appear (h). A sequence of two events was noted. Crack(s) in the tablets were often followed by more drastic modification of matrix structure, bursting being partial or total. The following terms have been employed: C1=crack type 1; nC1=multiple cracks type 1; C2=cracks type 2. C1 represents a single crack appearing along the radial surface of the cylinder. nC1 denotes multiple cracks appearing along the radial surface of the tablet. C2 means that one or more cracks appear on one or both facial surfaces of the tablet. The erosion process is not linked to the appearance of cracks. This allows the consideration of a rather semi-quantitative approach, keeping in mind that the more the tablets fully split apart, the higher are the risks of undesired burst release in vivo.

### Example 16b

### Evaluation of Swollen Tablet Integrity: Results

Table 6 shows that for an identical amount of electrolyte like NaCl, increasing non-electrolyte concentration improved the mechanical qualities of the swollen matrix. Indeed, for tablets containing 27.5% NaCl, cracks appeared after 7 h of immersion for 10% acetaminophen concentration compared to 10 h for 20% acetaminophen. Finally, they did not appear at all when acetaminophen concentration was elevated to 40%. This confirms that SD HASCA stabilized by an electrolyte can be used to formulate sustained drug-release matrices.

TABLE 6
Influence of drug-loading and NaCl content on the integrity of SD
HASCA swollen matrix tablets

| | |
|---|---|
| Formulation (% w/w) | Cracks |

| Drug | HASCA | NaCl | Time | Type | Erosion |
|---|---|---|---|---|---|
| 10 | 75 | 15 | 5.0/6.5 | C1/C2 | No |
| 10 | 62.5 | 27.5 | 7.0 | C2 | No |
| 10 | 55 | 35 | 5.0 | C2 | No |
| 10 | 45 | 45 | 5.0/8.0 | C1/C2 | + |
| 10 | 40 | 50 | 6.5/8.0 | C2/C1 | ++ |
| 20 | 52.5 | 27.5 | 10.5 | C2 | No |
| 20 | 45 | 35 | 6.0 | C2/C1 | No |
| 40 | 32.5 | 27.5 | No | No | No |

### Example 17

### Aspect of Typical SD HASCA Matrices

Tablets containing 40% of acetaminophen as model drug, 27.5% of NaCl and 32.5% of SD HASCA (manufactured as described in Example 9) were prepared as described in Example 10 to investigate the macroscopic aspects of SD HASCA matrix tablets after immersion in a pH gradient simulating the pH evolution of the gastrointestinal tract (pH 1.2 for 1 hour, pH 6.8 for 3 hours, and pH 7.4 until the end of the test). They weighed 600 mg each and were subjected to a 2.5 tons/cm² CF for 30 s.

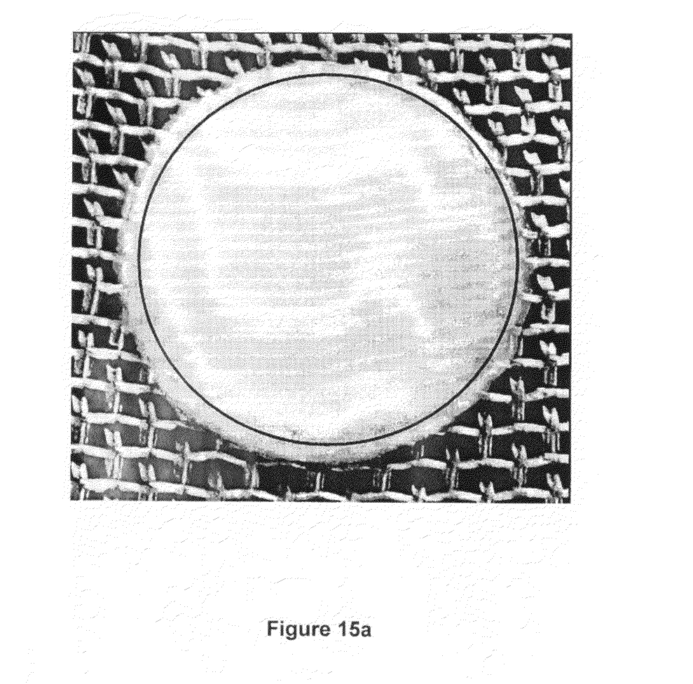

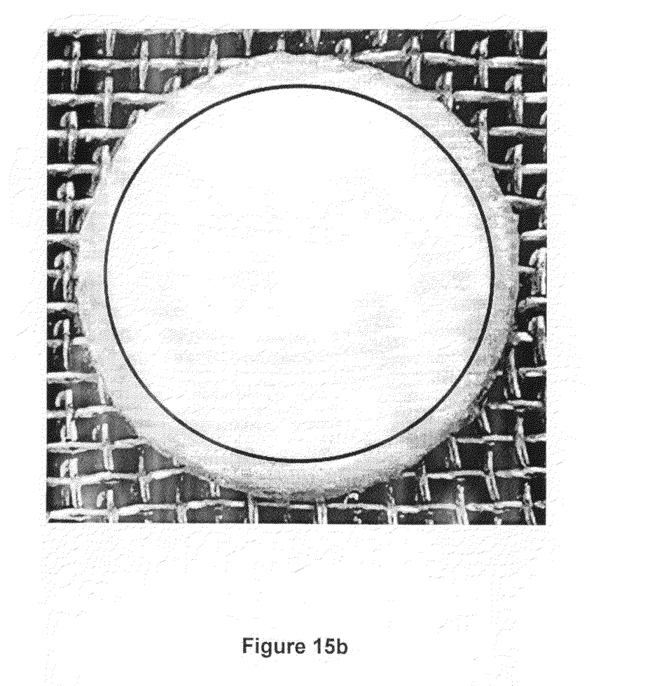

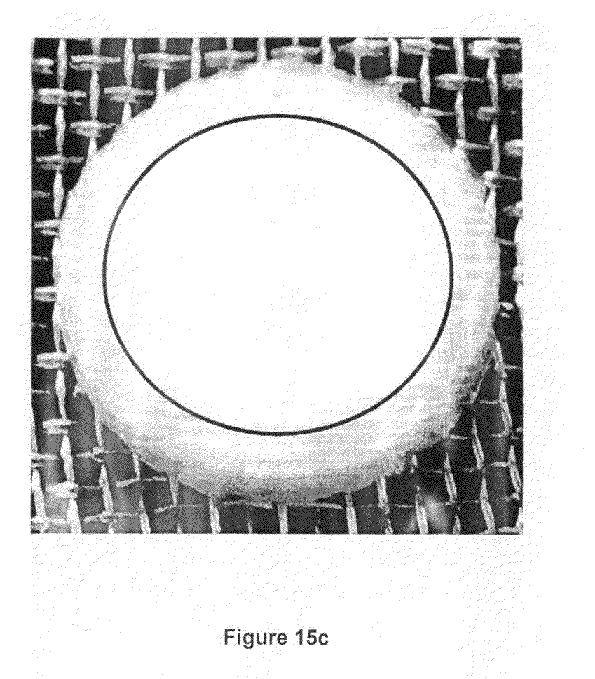

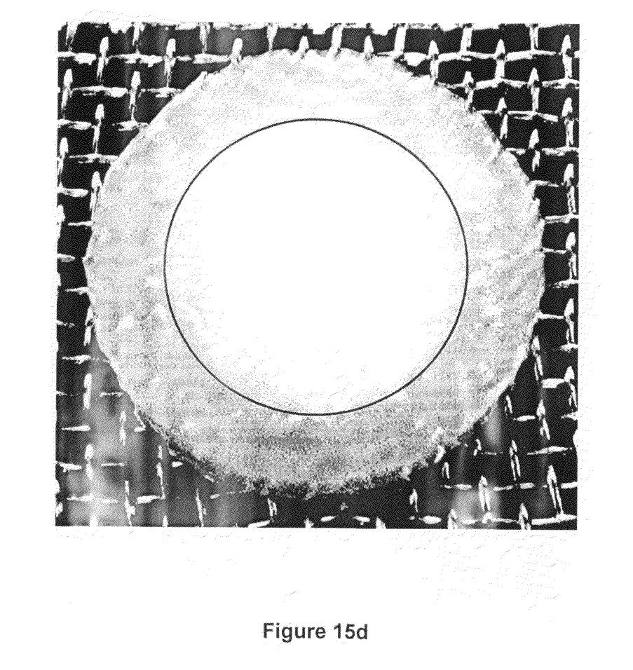

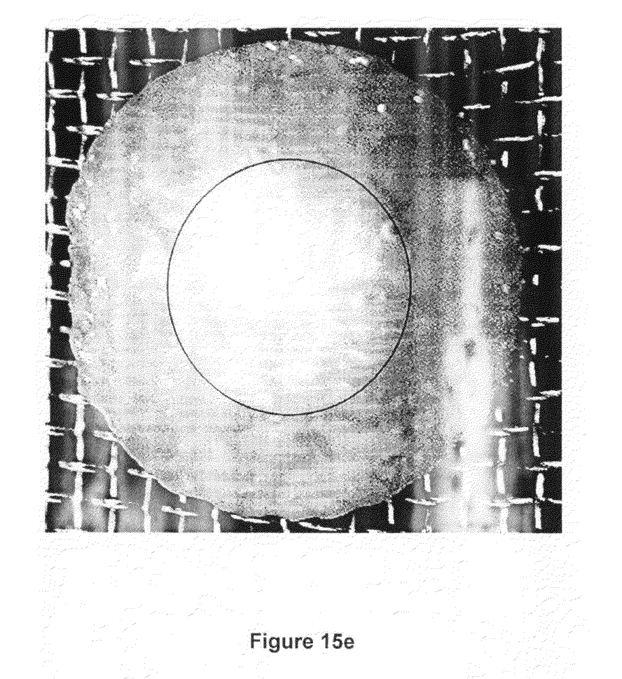

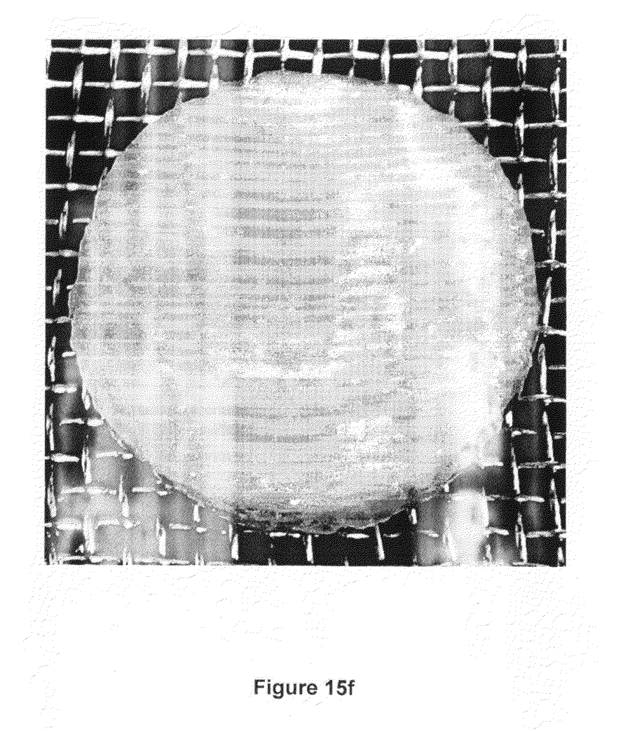

FIG. 15, from (a) to (f), present pictures of the above mentioned SD HASCA tablet matrices after immersion in the pH gradient simulating the pH evolution of the gastrointestinal tract: a) 2 hours of immersion b) 4 hours of immersion c) 8 hours of immersion d) 13 hours of immersion e) 16 hours of immersion and f) 22 hours of immersion. SD HASCA forms slowly and progressively a gel when combined with the right amount of electrolyte and drug in a matrix tablet. The tablet does not erode and does not crack. Hydrated SD HASCA matrices manifest rather moderate swelling, especially when compared to other typical hydrophilic matrices.

### Example 18

### Formulating SD HASCA Matrix Tablets with Electrolytes

Spray-dried HASCA was prepared in the same conditions as batch SD-A described in Example 2 using the materials described in Example 1. SD HASCA tablet matrices weighing 500 mg and compressed at 2.5 tons were obtained as described in Example 8a using the following formulations: A) acetaminophen 30%, HASCA 70% B) acetaminophen 30%, HASCA 55%, NaCl 15% C) acetaminophen 30%, HASCA 55%, KCl 15%. The sustained drug-release evaluation was performed in triplicate in conditions similar to the ones described in Example 8b except that the tablets were immersed for 30 min in an acidic medium (pH=1.2), then transferred to a phosphate buffer solution (pH=6.8) until the end of the test.

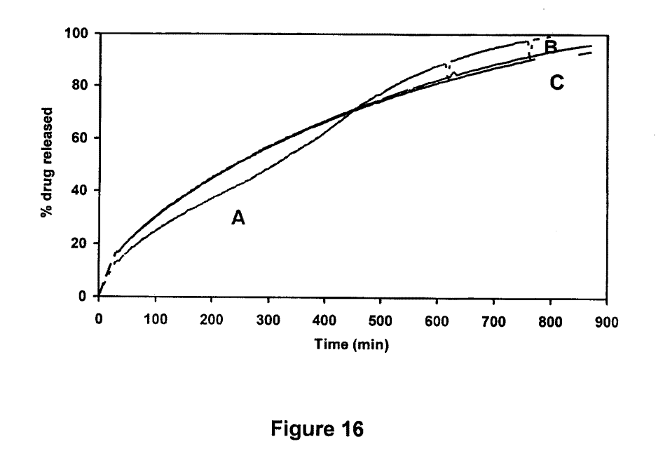

FIG. 16 shows the cumulative percentage of acetaminophen released in vitro in a pH gradient medium from the SD HASCA tablet matrices described above (A: Acetaminophen 30%, HASCA 70%; B: Acetaminophen 30%, HASCA 55%, NaCl 15%; C: Acetaminophen 30%, HASCA 55%, KCl 15%). Thus, other electrolytes than NaCl can be used with SD HASCA to formulate matrix tablets. FIG. 16 shows that the addition of the same quantity of sodium chloride or potassium chloride allows to maintain the integrity of the matrix tablets and control the drug-release better than in their absence. A longer sustained drug-release can be observed for tablets containing NaCl or KCl. More, the sudden acceleration of release rate around 300-400 minutes in the case of the tablet without electrolyte corresponds to a major crack appearing in the tablet. Such problems were not observed in the tablets containing NaCl or KCl.

### Example 19

### Varying HASCA Manufacturing Conditions

Spray-dried HASCA was prepared in the same conditions as batch SD-D described in Example 2 using the materials described in Example 1. The only difference in the manufacturing conditions was that the temperature of the spray-drier was set at 160° C. in place of 140° C.

A hardness control was performed according to the method described in Example 7a on 200 mg SD HASCA tablets (Ø: 12.6 mm, F: 2.5 tons, time of compression: 30 seconds): 22.2±0.4 SC (triplicate).

### Example 20

### Varying HASCA Manufacturing Conditions

Spray-dried HASCA was prepared in the same conditions as batch SD-D described in Example 2 using the materials described in Example 1. The only difference in the manufacturing conditions was that the speed of the pump of the spray-drier was set at 2 in place of 5.

A hardness control was performed according to the method described in Example 7a on 200 mg spray-dried HASCA tablets (□: 12.6 mm, F: 2.5 tons, time of compression: 30 seconds): 21.3±1.3 SC (triplicate).

### Example 21

### Varying Organic Solvent and High-Amylose Starch Type in SD HASCA Production

Materials are the same as those described in Example 1 except that a) isopropanol is used in place of ethanol b) two different types of amorphous pregelatinized HASCA provided in powder form by Roquette Frères (Lestrem, France), were tested:
1. Pregelatinized amorphous HASCA obtained from EURYLON VII (=P7), a special type of starch containing approximately 70% of amylose and 30% of amylopectin.
2. Pregelatinized amorphous HASCA obtained from EURYLON VI (=P6), a special type of starch containing approximately 60% of amylose and 40% of amylopectin.
   For each batch, the substitution degree was the same, i.e. 0.045.

Suspensions consisting in 10 g of amorphous pregelatinized HASCA and 80 g of a hydro-alcoholic solution (containing 83.58 % p/p water/isopropanol) were heated at a temperature of 70° C. The solution was kept at this temperature during 1 hour under stirring. At this time, the solution was cooled down under stirring until 35° C. A volume of pure isopropanol, corresponding to a final isopropanol to starch ratio of 3.2 w/w, was added “slowly, gradually” to the solution. The final suspension was passed in a Büchi B-190 Mini Spray Drier™ (Flawill, Switzerland) at a temperature of 140° C. to obtain HASCA in form of a fine dry powder. The spray-drier airflow was 601 NormLitre/Hour.

Table 7 a & b describes the composition of HASCA suspensions during the two main operational steps, i.e. heating of the initial hydro-alcoholic suspensions and spray-drying of the final suspensions where % w/w WATER=the percent by weight of water in the starting hydro-alcoholic solution in which the powder is dispersed at the beginning of the process. 80 g of this solution are used to disperse each HASCA powder sample.

SOLUTION weight (g)=weight of hydro-alcoholic solution used to disperse each HASCA powder sample.
HASCA weight (g)=weight of HASCA powder added to the hydro-alcoholic solution.
% w/w HASCA-I=[HASCA weight/(HASCA weight+SOLUTION weight)]*100
% w/w water-I=[(water weight)/(HASCA weight+SOLUTION weight)]*100.
% w/w Isop-I=[(Isopropanol weight)/(HASCA weight+SOLUTION weight)]*100.
Isop added (g)=quantity (g) of isopropanol added to the hydro-alcoholic suspension to obtain a spray-drying suspension having a isop/HASCA-II ratio of 3.2.
Isop/HASCA-II=3.2=ratio of the total weight of isopropanol on the weight of HASCA in the suspension to be spray-dried.
% w/w HASCA-II=[HASCA weight/(HASCA weight+SOLUTION weight+Isopropanol added)]*100
% w/w water-II=[water weight/(HASCA weight+SOLUTION weight+Isopropanol added)]*100
% w/w Isop-II=[Isopropanol total weight/(HASCA weight+SOLUTION weight+Isopropanol added)]*100

TABLE 7
Compositions of a) the HASCA initial hydro-alcoholic suspensions
(heating step) and b) the spray-drying suspensions (drying step)
a) Initial hydro-alcoholic suspension

| | | | HASCA | | | |
|---|---|---|---|---|---|---|
| | % w/w | SOLUTION | weight | % w/w | % w/w | % w/w |
| Batch | WATER | weight (g) | (g) | HASCA-I | water-I | isop-I |
| P7 | 83.58 | 80 | 10 | 11.11 | 74.29 | 14.60 |
| P6 | 83.58 | 80 | 10 | 11.11 | 74.29 | 14.60 |

b) Spray-drying suspension

| | isop | | | | |
|---|---|---|---|---|---|
| | added | % w/w | % w/w | % w/w | isop/ |
| Batch | (g) | HASCA-II | water-II | isop-II | HASCA-II |
| P7 | 18.64 | 9.21 | 61.55 | 29.25 | 3.2 |
| P6 | 18.64 | 9.21 | 61.55 | 29.25 | 3.2 |

### Example 22

### Testing SD HASCA Tablet Hardness

SD HASCA tablets weighing 200 mg were prepared by direct compression. The excipient, obtained as described in Example 21 (isopropanol), was compressed in a hydraulic press (Workshop Press PRM 8 type, Rassant Industries, Chartres, France) at a compaction load of 2.5 tons/cm² with a dwell time of 30 s (flat-faced punch die set). The diameter of all the tablets was 12.6 mm. Tablet hardness (Strong-Cobs or SC) was quantified with a hardness tester (ERWEKA® Type TBH 200, Erweka Gmbh, Heusenstamm, Germany). The data presented here are the mean values of three measurements.

The results are presented in Table 8. It is concluded from Tables 7 and 8 that not only can SD HASCA powders be obtained using isopropanol and starch containing lower amounts of amylose, i.e. 60%, but also that such SD HASCAs obtained following the process described above lead to good tablet strength.

TABLE 8
Hardness determined for four 200 mg tablets (Ø = 12.6 mm,
F = 2.5 tons/cm²) of pure SD HASCA

| | Mean ± SD |
|---|---|
| HASCA type | (Strong-Cobbs) |
| P7 | 17.8 ± 2.3 |
| P6 | 15.2 ± 1.9 |

### Example 23

### Testing SD HASCA Tablet Sustained Drug-Release Properties: Effect of Changing the Organic Solvent Used in the Manufacturing Process

SD HASCA tablet matrices weighing 600 mg and compressed at 2.5 tons were obtained as described in Example 8a using the following formulations: 40% acetaminophen, 27.5% NaCl and P7 SD HASCA (obtained as described in Example 21) ad 100%. The sustained drug-release evaluation was performed in triplicate in conditions similar to the ones described in Example 8b except that the tablets were immersed for 30 min in an acidic medium (pH=1.2), then transferred to a phosphate buffer solution (pH=6.8) until the end of the test.

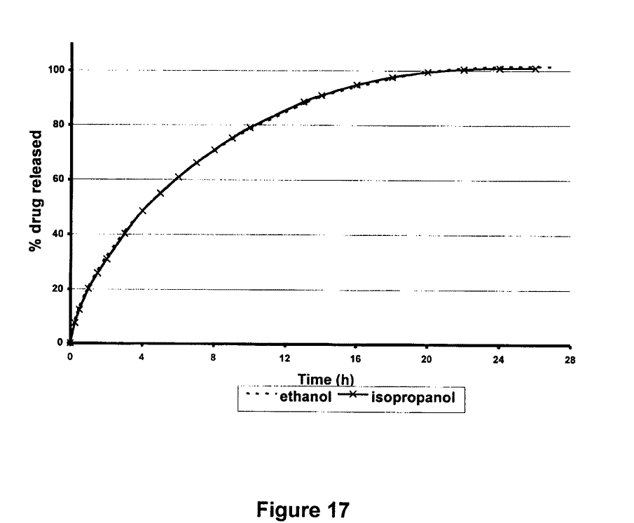

FIG. 17 shows the effect of the solvent used in the spray-drying process on % acetaminophen release from 600-mg P7 SD HASCA matrix tablets containing 40% acetaminophen and 27.5% NaCl (dotted line=ethanol; continuous line=isopropanol). The samples obtained with ethanol as organic solvent were obtained in conditions similar to the ones described for isopropanol and described in Example 21. Changing ethanol for isopropanol in the heating and spray-drying processes did not affect the sustained drug-release properties of SD HASCA tablets. Ethanol can be advantageously replaced by isopropanol. Using isopropanol in place of ethanol has been generally recognized as cheaper and safer regarding spray-drying manufacturing processes.

### Example 24

### Testing SD HASCA Tablet Sustained Drug-Release Properties: Effect of Changing the High-Amylose Starch Used in the Manufacturing Process

SD HASCA tablet matrices weighing 600 mg and compressed at 2.5 tons were obtained as described in Example 8a using the following formulations: 40% acetaminophen, 22.5 or 27.5% NaCl and P6 SD HASCA (obtained as described in Example 21) ad 100%. The sustained drug-release evaluation was performed in triplicate in conditions similar to the ones described in Example 8b except that the tablets were immersed for 30 min in an acidic medium (pH=1.2), then transferred to a phosphate buffer solution (pH=6.8) until the end of the test.

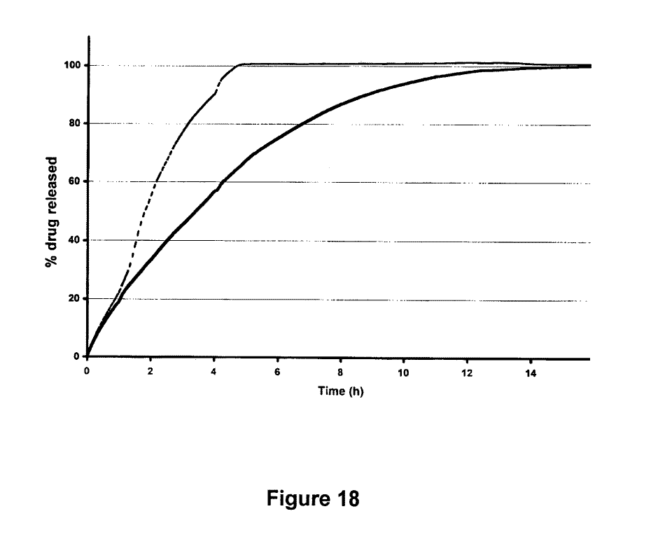

FIG. 18 shows the effect of NaCl content on % acetaminophen release from 600-mg P6 SD HASCA matrix tablets containing 40% acetaminophen (dotted line=27.5% NaCl; continuous line=22.5% NaCl). Note that P6 SD HASCA is obtained by spray-drying an amorphous pregelatinized HASCA obtained from EURYLON™ VI. Spray-dried HASCA obtained from Eurylon™ VI allows obtaining sustained drug-release tablets. It appears that decreasing amylose content accelerates the drug-release but lowering the electrolyte amount can decrease the drug-release rate to compensate that effect.

### Example 25

### Testing SD HASCA Tablet Sustained Drug-Release Properties: Effect of Changing the High-Amylose Starch Used in the Manufacturing Process

SD HASCA tablet matrices weighing 500 mg and compressed at 2.5 tons were obtained as described in Example 8a using the following formulations: 40% acetaminophen, 17.5% NaCl and P6 SD HASCA (obtained as described in Example 21) ad 100%. The sustained drug-release evaluation was performed in triplicate in conditions similar to the ones described in Example 8b except that the tablets were immersed for 30 min in an acidic medium (pH=1.2), then transferred to a phosphate buffer solution (pH=6.8) until the end of the test.

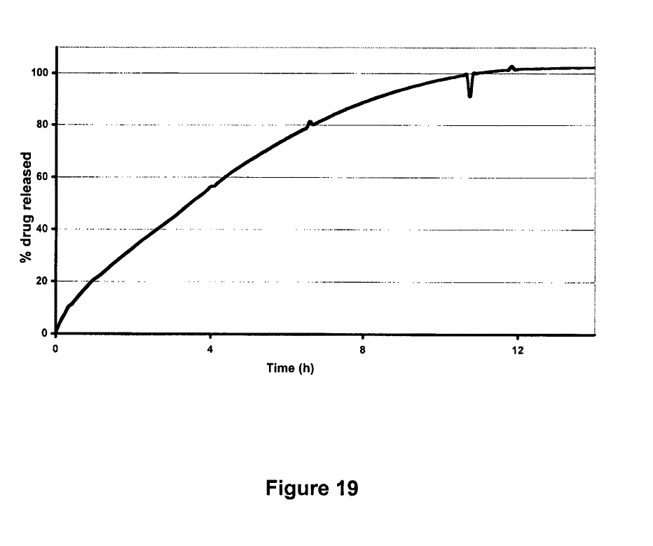

FIG. 19 shows the % acetaminophen release from 500-mg P6 SD HASCA matrix tablets containing 40% acetaminophen and 17.5% NaCl. Note that P6 SD HASCA is obtained by spray-drying a pregelatinized amorphous HASCA obtained from EURYLON VI. Substituted amylose is known to decrease its total drug-release time in function of the tablet weight. It is shown here that the loss in total drug-release time due to the decrease in tablet weight can be compensated by a decrease in NaCl content (see also FIG. 18). Thus, SD HASCA can be composed of a lower proportion of amylose compared to the starch starting material described until now in U.S. Pat. No. 5,879,707 and Canadian Patent Application No. 2,591,806 though it is obvious that one still needs a starch with a high content in amylose.

While specific embodiment of the present invention have been described and illustrated, it will be apparent to those skilled in the art that numerous modifications and variations can be made without departing from the scope of the invention.

1. A process for obtaining a spray-dried high amylose sodium carboxymethyl starch comprising a major fraction of amorphous form and optionally a minor fraction of crystalline V form, said process comprising the following steps:
a) providing an amorphous pregelatinized high amylose sodium carboxymethyl starch;
b) dispersing the amorphous pregelatinized high amylose sodium carboxymethyl starch in a solution comprising water and at least one first pharmaceutically acceptable organic solvent miscible with water and suitable for spray-drying; and
c) spray-drying the dispersion to obtain the spray-dried high amylose sodium carboxymethyl starch comprising a major fraction of amorphous form and optionally a minor fraction of crystalline V form, in the form of a powder.

2. The process of claim 1, wherein the uncross-linked amorphous pregelatinized high amylose sodium carboxymethyl starch provided in step a) is dried by a roller-dryer.

3. The process of claim 1, wherein the at least one first organic solvent is ethanol, isopropanol or any mixture thereof.

4. The process of claim 1, wherein an amount of a second pharmaceutically acceptable organic solvent miscible with water, which is different or identical to the at least one first organic solvent, is added to the dispersion before the spray-drying step c).

5. The process of claim 4, wherein the at least one first and second organic solvents, which are different or identical, are ethanol, isopropanol or any mixture thereof.

6. The process of claim 1, wherein in step a) the water to organic solvent(s) weight ratio is above 1.

7. The process of claim 1, wherein the uncross-linked amorphous pregelatinized high amylose sodium carboxymethyl starch comprises at least about 50 weight % of amylose and has a substitution degree comprised between about 0.005 and about 0.070.

8. A spray-dried high amylose sodium carboxymethyl starch sustained-release excipient comprising a major fraction of amorphous form and optionally a minor fraction of crystalline V form, characterized in that it is obtained by the process of claim 1.

9. A spray-dried high amylose sodium carboxymethyl starch sustained-release excipient comprising a major fraction of amorphous form and optionally a minor fraction of crystalline V form, said excipient being obtained by spray-drying a dispersion of an uncross-linked amorphous pregelatinized high amylose sodium carboxymethyl starch in a solution comprising water and ethanol, or isopropanol or a mixture thereof, said uncross-linked amorphous pregelatinized high amylose sodium carboxymethyl starch comprising at least about 60 weight % of amylose and having a substitution degree of about 0.045.

10. Use of the spray-dried high amylose sodium carboxymethyl starch sustained-release excipient as defined in claim 8 in the preparation of a tablet for sustained-release of at least one drug.

11. A tablet for sustained-release of at least one drug comprising the spray-dried high amylose sodium carboxymethyl starch sustained-release excipient as defined in claim 8 and at least one drug.

12. The tablet of claim 11 further comprising at least one electrolyte.

13. The tablet of claim 12, wherein the electrolyte is another excipient, another drug or a mixture thereof.
